(19) United States
(12) Patent Application Publication
(10) Pub. No.: US 2024/0124938 A1
(43) Pub. Date: Apr. 18, 2024

(54) VISUAL DETECTION OF PBD INDUCED DNA CROSSLINKS

(71) Applicants: INSERM (INSTITUT NATIONAL DE LA SANTÉ ET DE LA RECHERCHE MÉDICALE), PARIS (FR); INSTITUT JEAN PAOLI & IRENE CALMETTES, Marseille (FR); AIX-MARSEILLE UNIVERSITÉ, MARSEILLE (FR); CENTRE NATIONAL DE LA RECHERCHE SCIENTIFIQUE CNRS, PARIS (FR)

(72) Inventors: Christophe LACHAUD, Marseille Cedex 09 (FR); Sébastien COMBES, Marseille Cedex 5 (FR); Sébastien ABEL, Marseille Cedex 5 (FR); Gilles AUDOLY, Marseille Cedex 09 (FR); Sara BERRADA, Marseille Cedex 09 (FR)

(21) Appl. No.: 17/768,642

(22) PCT Filed: Oct. 16, 2020
(86) PCT No.: PCT/EP2020/079233

§ 371 Date: Apr. 13, 2022

(30) Foreign Application Priority Data
Oct. 16, 2019 (EP) 19306349.2

(51) Int. Cl. C12Q 1/6886; C07C 205/59; C07D 207/22; C07D 487/04; C12Q 1/6841

(52) U.S. Cl. CPC C12Q 1/6886; C07C 205/59; C07D 207/22; C07D 487/04; C12Q 1/6841; C12Q 2600/106; C12Q 2600/156

(57) ABSTRACT

The present invention relates to the field of oncology. laboratory tools and methods, and especially anti-tumor DNA crosslinking agents. Most patients with advanced solid tumors develop resistance to chemotherapy due to the ability of cancer cells to repair or tolerate sustained DNA damages. The inventors showed that the compounds according to the present invention allow the detection and visualization of alkylated DNA damages induced by PBDs without altering their DNA crosslinking ability. This enables the study of the effect and properties of PBDs. In particular, the present invention relates new derivates of PBD molecules and their synthesis. The present invention also relates to a method for visualizing DNA crosslinking: to a method for assessing the resistance of a tumor to a crosslinking agent and to a method for identifying a molecule or treatment for improving the efficiency of a crosslinking agent.

### FIELD OF THE INVENTION

The present invention relates to the field of oncology, laboratory tools and methods. More particularly, it provides new compounds suitable for visualizing DNA crosslinks.

### BACKGROUND OF THE INVENTION

The pyrrolo[2,1-c][1,4]benzodiazepines (PBDs) are sequence-selective DNA minor-groove binding antitumor agents which are based on the naturally occurring anthramycin, which was isolated from Streptomyces refuineus in 1965.

The skeletal structure of the PBDs contains (i) a substituted aromatic A-ring, (ii) a diazepine B-ring and (iii) a pyrrolidine C-ring, with an S-chiral center at the C11a-position between the B and C rings. This provides a 3-dimensional shape perfectly crafted for the molecules to fit within the DNA minor groove. They also possess an electrophilic imine moiety (or equivalent carbinolamine or carbinolamine methyl ether) within their B-ring which can form a covalent amine linkage between their C11 carbon and the C2-NH₂ group of a guanine base.

Since the discovery of anthramycin, many synthetic PBDs have been developed. In particular, monomeric PBD units have been joined together through their C7/C7′- and C8/C8′-positions to afford PBD dimers. The unique structure of the PBD dimers which contain two alkylating imine functionalities allows them to form interstrand or intrastrand DNA crosslinks in addition to mono-alkylated adducts, thus resulting in greater DNA stabilization compared to monomeric PBDs (S. J. Gregson et al. J. Med. Chem. 2001, 44, 737-748; S. J. Gregson et al. J. Med. Chem. 2004, 47, 1161-1174). Because DNA interstrand crosslinks (ICLs) block essential aspects of DNA metabolism, such as replication and transcription, they are highly cytotoxic. PBD dimers generally have significantly greater cytotoxicity, antitumor activity and antibacterial activity compared to PBD monomers. Once covalently bound to DNA, PBD dimers have been shown to mediate in cells a consequent number of biological effects, such as DNA strand breakage (J. M. Reid et al. Cancer Chemother. Pharmacol. 2011, 68, 777-786), inhibition of DNA processing enzymes (for example endonuclease BamH1) and specific transcription factors (for example NF-KB). They have also been shown to modulate various signaling pathways, and in particular p53-dependent and -independent apoptogenic signaling (Y.-W. Chou et al., Eur. J. Med. Chem. 2016, 109, 59-74). Such properties could, in part, explain the anticancer activity of the PBDs in animal models and humans.

Recent evolution has been to use PBD dimer analogues attached to tumor-targeting antibodies to create antibody-drug conjugates (ADCs). A number of them are now in phase 3 clinical trials, with many others in pre-clinical development.

The best known PBD dimer, named SJG-136, has recently completed Phase II clinical trials in patients with leukemia and ovarian cancer. SJB-136 is a rationally designed pyrrolobenzodiazepine dimer having the following formula:

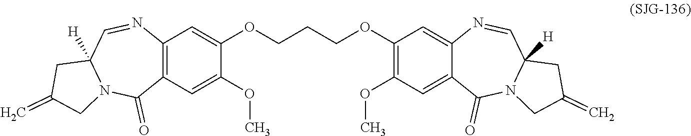

that binds in the minor groove of DNA. It spans 6 bp with a preference for binding to purine-GATC-pyrimidine sequences. SJG-136 has demonstrated potent activity in a range of cell lines and tumor xenograft models (Hartley J A, et al. Cancer Research. 2004; 64:6693-6699; Alley M C, et al. Cancer Research. 2004;64:6700-6706).

However, as for many available anti-tumor DNA crosslinking agents, such as cisplatin for example, most patients with advanced solid tumors develop resistance to chemotherapy due to the ability of cancer cells to repair or tolerate the sustained DNA damages.

Accordingly, there remains a need in the art for a mean allowing to visualize the DNA-PBDs crosslinks in the study of the effect and properties of already known and yet to be tested PBDs.

Moreover, there remains a need for a mean to visualize the DNA-PBDs crosslinks while testing compound candidates that could help improve the properties of the said PBDs and/or reduce the above-mentioned resistance of tumor cells. There accordingly remains a need for a method for screening compounds that can be used in combination with PBD dimers to reduce the resistance of tumor cells to said PBD dimers, and thus for screening compounds which, when used in combination with PBD dimers, improves their anti-tumoral properties.

There is moreover a need for a method or a tool for quantifying and/or qualifying PBD dimers binding to DNA.

Furthermore, all these needs must be met without altering the DNA crosslinking ability of the considered PBDs, i.e. without reducing or even abolishing the DNA crosslinking ability of the considered PBDs.

According to the present invention, the terms “PBD” and “PBD dimer” are used interchangeably in order to designate PBD dimers, and in particular PBD dimers of formula (I).

### SUMMARY OF THE INVENTION

The inventors have developed new compounds mimicking the properties of PBDs dimers by creating DNA-crosslinks, while being detectable, thereby enabling detection of DNA crosslinks lesions due to crosslinking agents, and in particular to PBD dimers, in cells. The compounds of the invention can accordingly be useful for screening or identifying molecules and/or treatments which allow, for example:
improving the ability of crosslinking agents, and in particular of PBD dimers, to create DNA cross-links in a cell;
prevent or delay the occurrence of resistance to crosslinking agents, and in particular to PBD dimers; and/or
overcoming or reducing resistance to crosslinking agents, and in particular to PBD dimers.

The compounds of the invention can in consequence be useful for screening or identifying compounds and/or treatments to be used in combination with crosslinking agents, and in particular with PBD dimers.

Accordingly, the present invention relates to a compound of formula (I):

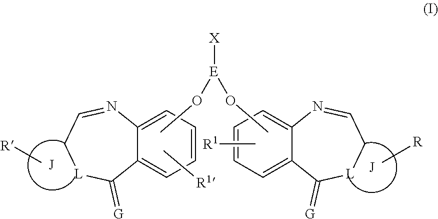

wherein:
X is a C₂-C₇ azide or alkyne group,
E is a C₃-C₁₂ alkyl group, or a group of the following formula

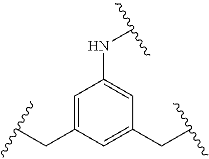 [SMILES: *Cc1cc(C*)cc(N*)c1]

wherein the label ⋆ identifies the bond to the radical X;
G is an oxygen atom or CH₂ group,
L is a nitrogen atom or a CH group,
J is a saturated or unsaturated, mono- or polycondensed C₅-C₆ heterocycloalkyl group containing nitrogen atom
R is selected from the group consisting in hydrogen atom, halogen atom and in particular a fluorine atom, C₂-C₃ alkynyl group and in particular a propynyl group, substituted by a secondary amine, methylidene group, C₁-C₃ alkenyl group optionally substituted by one or several halogen atoms in particular by fluorine atoms, or a secondary amine, C₁-C₃ alkyl group, phenyl group and carbonyl group,
R¹ is selected from the group consisting in hydrogen atom, C₁-C₃ alkyl group, C₁-C₃ alkoxy group optionally substituted by a secondary amine or an ethynyloxy group, and
wherein G′, J′, n′, l′, R′ and R¹′ are independently chosen from the same group as their corresponding counterparts G, J, n, l, R and R¹.

In a particular embodiment, the present invention relates to a compound of formula (II):

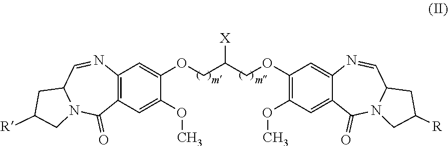

wherein:
m′ and m″ are integers independently ranging from 1 to 10 and their sum is less than 12, X, R and R′ are as defined above.

In a more particular embodiment, the invention pertains to a compound of formula (III):

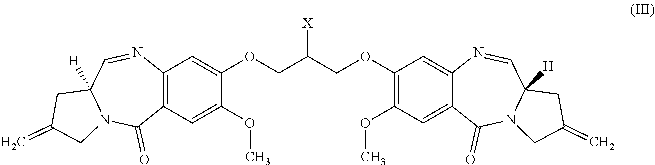

wherein X is as defined above.

In a particular embodiment, X is a prop-2-ynyl group.

The present invention also relates to a kit comprising:
at least one compound of formula (I) according to the invention; and
at least one label bearing a group complementary to the X radical of the compounds of formula (I) for a “click-chemistry” reaction, wherein the label is preferably a fluorescent label or a biotinylated label.

Furthermore, the present invention relates to the in vitro or ex vivo use of a compound according to the present invention, or of a kit as provided herein, as a research tool, in particular for visualizing DNA crosslinks induced in a cell by the compounds of formula (I) of the invention.

The present invention accordingly further pertains to an in vitro or ex vivo method for visualizing DNA crosslinks in a cell, the method comprising:
(a) having at least one cell,
(b) contacting said cell with at least one compound of formula (I) according to the invention under conditions allowing said compound to induce DNA crosslinks in the cell,
(c) contacting the cell obtained in step (b) with at least one label bearing a group which is complementary for a click-chemistry reaction to the X radical of the compound of formula (I) under conditions allowing the reaction of click-chemistry between the said X radical and the complementary group, and
(d) detecting the label in the cell obtained at step (c).

The present invention also relates to an in vitro or ex vivo method for assessing the resistance or sensitivity of a tumor in a patient to a crosslinking agent, and in particular to a PBD dimer, more particularly to a compound of formula (I) according to the invention, comprising at least the steps of:
(a) having at least one cell from the said tumor;
(b) contacting said cell with at least one compound of formula (I) according to the invention under conditions allowing said compound to induce DNA crosslinks in the cell;
(c) contacting the cell obtained in step (b) with at least one label, preferably a fluorescent label, bearing a group which is complementary for a click-chemistry reaction to the X radical of the compound of formula (I) under conditions allowing the reaction of click-chemistry between the said X radical and the complementary group;
(d) measuring the labelling in the cell obtained at step (c); and
(e) optionally comparing the labelling measured at step (d) to a reference level.

The present invention also relates to an in vitro or ex vivo method for assessing the resistance or sensitivity of a tumor in a patient to a crosslinking agent bearing a X radical as defined above, and in particular to a PBD dimer bearing a X radical as defined above, more particularly to a compound of formula (I) according to the invention and preferably to a compound of formula (II) according to the invention, comprising at least the steps of:
(a) having at least one cell from the said tumor;
(b) contacting said cell with at least one said crosslinking agent, in particular a PBD dimer, notably a compound of formula (I) according to the invention and preferably a compound of formula (II) according to the invention under conditions allowing said compound to induce DNA crosslinks in the cell;
(c) contacting the cell obtained in step (b) with at least one label, preferably a fluorescent label, bearing a group which is complementary for a click-chemistry reaction to the X radical of the crosslinking agent, in particular of the PBD dimer, notably of the compound of formula (I), preferably of formula (II), under conditions allowing the reaction of click-chemistry between the said X radical and the complementary group;
(d) measuring the labelling in the cell obtained at step (c); and
(e) optionally comparing the labelling measured at step (d) to a reference level.

In such a method, it can be understood that the higher the labelling of the cell, the higher its sensitivity to a crosslinking agent, in particular to a PBD dimer and more particularly to a compound of formula (I) according to the invention and, inversely, the lower the labelling of the cell, the higher its resistance to a crosslinking agent, in particular to a PBD dimer and more particularly to a compound of formula (I) according to the invention.

Furthermore, the present invention relates to an in vitro or ex vivo method for identifying or screening a candidate molecule and/or a candidate treatment for its ability to improve the efficiency of a crosslinking agent, in particular of a PBD dimer, and more particularly of a PBD dimer of formula (I) of the invention, comprising at least the steps of:
(a) having at least one cell, preferably a tumor cell;
(b) contacting said cell with the candidate molecule and/or applying the candidate treatment to said cell;
(b′) contacting the cell with at least one compound of formula (I) according to the invention, under conditions allowing said compound to induce DNA crosslinks in the cell;
(c) contacting the cell obtained after the previous steps with at least one label, preferably a fluorescent label, bearing a group which is complementary to the X radical of the compound according to the invention, under conditions allowing the reaction of click-chemistry between the X radical and the complementary group;
(d) measuring the labelling in the cell obtained at step (c); and
(e) comparing the intensity of the labelling obtained at step (d) to a reference labelling intensity obtained when the method is performed in the absence of the candidate molecule and/or candidate treatment;
wherein steps (b) and (b′) can occur simultaneously or sequentially, preferably in that order.

Furthermore, the present invention relates to an in vitro or ex vivo method for identifying or screening a candidate molecule and/or a candidate treatment for its ability to improve the efficiency of a crosslinking agent bearing a X radical as defined above, in particular of a PBD dimer bearing a X radical as defined above, more particularly of a PBD dimer of formula (I) of the invention and preferably of a compound of formula (II) according to the invention, comprising at least the steps of:
(a) having at least one cell, preferably a tumor cell;
(b) contacting said cell with the candidate molecule and/or applying the candidate treatment to said cell;
(b′) contacting the cell with at least one crosslinking agent, in particular a PBD dimer, notably a compound of formula (I) according to the invention and preferably a compound of formula (II) according to the invention, under conditions allowing said compound to induce DNA crosslinks in the cell;
(c) contacting the cell obtained after the previous steps with at least one label, preferably a fluorescent label, bearing a group which is complementary to the X radical of the crosslinking agent, in particular of the PBD dimer, notably of the compound of formula (I) according to the invention and preferably of a compound of formula (II) according to the invention, under conditions allowing the reaction of click-chemistry between the X radical and the complementary group;
(d) measuring the labelling in the cell obtained at step (c); and
(e) comparing the intensity of the labelling obtained at step (d) to a reference labelling intensity obtained when the method is performed in the absence of the candidate molecule and/or candidate treatment;
wherein steps (b) and (b′) can occur simultaneously or sequentially, preferably in that order.

In such a method, it can be understood that, a difference between both labeling is representative of an effect of the candidate molecule and/or candidate treatment. In particular, the higher the labelling of the cell when a compound of formula (I) according to the invention is used in combination with the candidate molecule and/or candidate treatment when compared to a method implementing a compound of formula (I) according to the invention alone, the more said candidate molecule and/or candidate treatment improves the efficiency of crosslinking agents, in particular of PBD dimers and more particularly of PBD dimers of formula (I) according to the invention.

The present invention further provides a method for preparing a compound of formula (I) according to the invention. The present invention moreover provides a method for preparing intermediates of a compound of formula (I) according to the invention.

### BRIEF DESCRIPTION OF THE DRAWINGS

FIG. 1 represents confocal microscopy observations of samples of mice bone marrow in mouse femur tissues treated with 0.3 mg·kg⁻¹ of either SJG136 (top line of images) (corresponding to a compound outside of the invention) or SJG136-click (bottom line of images) (corresponding to the compound of formula (IV) according to the invention). The nucleus of the sample's cells are identified on the left column by DAPI staining. The middle column represents images obtained by exposing clickable 647 fluorophore (Alexa Fluor™ 647 azide, sold by the company Thermo Fisher) to the samples treated with SJG136 or SJG136-click to detect the SJG136 or SJG136-click. On the right column, observations from the left and middle column are merged. Some of the observable DNA crosslinking are indicated by white arrows in the middle and right cases of the bottom line.

### DETAILED DESCRIPTION OF THE INVENTION

The present invention relates to a compound useful for mimicking the properties of crosslinking agents, and in particular of PBDs dimers, by creating DNA crosslinks while being detectable.

As previously mentioned, PBDs are a family of sequence-selective DNA minor-groove binding agents that form a covalent aminal bond between their C11-position and the C2-NH₂ groups of guanine bases of DNA.

For example, the above-mentioned SJG-136 PBD dimer interacts with DNA as follows:

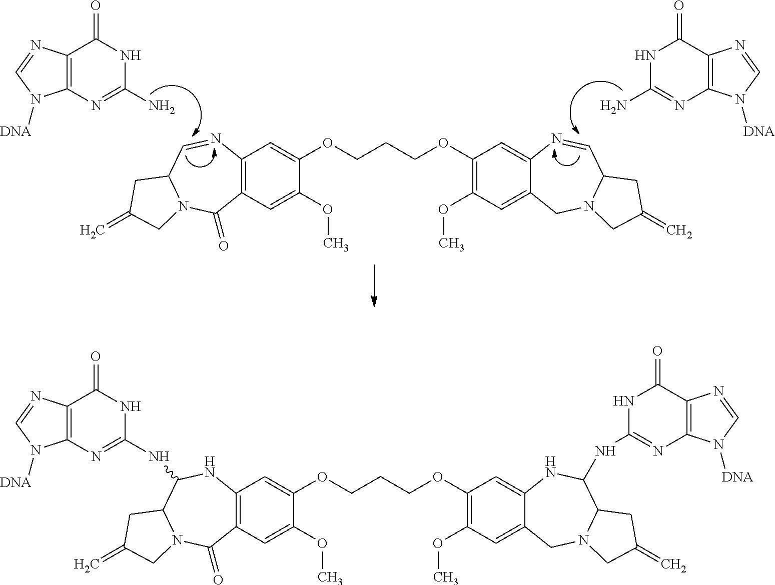

As previously stated, it has become more and more important to be able to monitor interactions of crosslinking agents with DNA since these compounds have shown a considerable interest in cancer research while resistance of cancer cells to such compounds has become a consequent problem in the medical field.

Unexpectedly, the inventors discovered that click chemistry can be used to perform a labelling of crosslinking agents, in particular of PBD dimers, while not altering the advantageous and sought properties of these compounds. In order to combine these requirements, it is however necessary that the group involved in the labelling in the crosslinking agent be specifically located.

The click-chemistry is a known chemical reaction also named azide-alkyne cycloaddition (AAC) as represented in following Scheme:

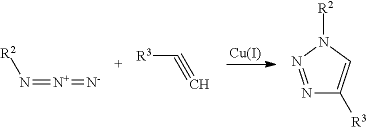

In the case of the instant invention:

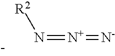

represents a compound of formula (I) of the invention and R³ represent a label, in particular a dye, preferably a fluorescent dye; or

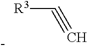

represents a compound of formula (I) of the invention and R² represent a label, in particular a dye, preferably a fluorescent dye.

According to a specific embodiment, this cycloaddition may be conducted in presence of a catalyzer, and is then named CuAAC, said catalyzer generally being a copper compound, and in particular a copper(I) compound. The copper(I) needed as catalyst in the CuAAC can be provided by the use of copper(II) precursors with a reducing agent, such as sodium ascorbate or p-hydrochinone for instance, by copper(I) salts or by pre-formed copper(I) complexes.

“Click-chemistry” reactions, and more particularly CuAAC are biocompatible, more particularly compatible with the presence of a plurality of biological entities and can be carried out in cells.

To the inventor's knowledge, using such technic to label crosslinking agents, in particular PBD dimers, to allow visualizing their interaction with DNA while not altering their crosslinking ability has never been performed. This cycloaddition has already been disclosed for labelling cisplatin, another family of drug known to interact with DNA (WO2017/102934). However, these platin-containing drugs are significantly different from PBD dimers. Furthermore, the localization of the group involved in the chemical click reaction is located in one end of the cisplatin molecule by contrast to the instant compounds of formula (I) requesting the localization of this same group in the central area of the molecules.

A “cell” according to the present invention is an isolated cell comprising DNA, in particular DNA in a double-stranded form. More particularly, a cell as mentioned in the present text can be a cancer cell, i.e. a cell from a cancer. Said cancer cell can either originate from a commercial cancer cell line or from a cancer in an individual, in particular in a human.

### Compounds of the Invention

As mentioned above, a compound according to the present invention has the following formula (I):

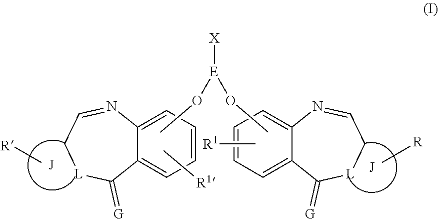

wherein:
X is a C₂-C₇ azide or alkyne group,
E is a C₃-C₁₂ alkyl group, or a group of the following formula

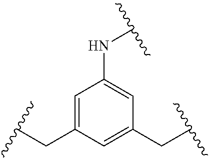 [SMILES: *Cc1cc(C*)cc(N*)c1]

wherein the label ⋆ identifies the bond to the radical X;
G is an oxygen atom or a CH₂ group,
L is a nitrogen atom or a CH group,
J is a saturated or unsaturated, mono- or polycondensed C₅-C₆ heterocycloalkyl group containing nitrogen atom
R is selected from the group consisting in hydrogen atom; halogen atom, in particular a fluorine atom; C₂-C₃ alkynyl group, in particular a propynyl group, substituted by a secondary amine; methylidene group; C₁-C₃ alkenyl group, optionally substituted by one or several halogen atom(s), and in particular by (a) fluorine atom(s), or a secondary amine; C₁-C₃ alkyl group; phenyl group and carbonyl group,
R¹ is selected from the group consisting in hydrogen atom, C₁-C₃ alkyl group, C₁-C₃ alkoxy group optionally substituted by a secondary amine or an ethynyloxy group, and
wherein G′, J′, R′ and R¹′ are independently chosen from the same group as their corresponding counterparts G, J, R and R¹.

In particular, G and G′ are oxygen atoms.

In a particular embodiment, the compound of formula (I) has a symmetry plane that encompasses the E—X bond. In a more particular embodiment, substituents R¹ and —O—E on the aromatic ring are in ortho positions relatively to each other. Preferably, R¹ and R¹′ are C₁-C₃ alkoxy groups, more preferably are methoxy groups.

In a specific embodiment, L is a nitrogen atom and J and J′ are C₅ heterocycloalkyl groups. Such compound corresponds to a compound of formula (II):

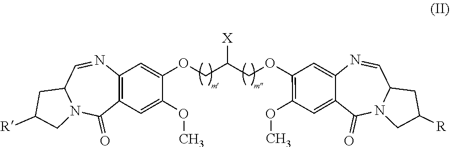

wherein:
m′ and m″ are integers independently ranging from 1 to 10 and their sum is less than 12,
X, R and R′ are as defined above.

In another specific embodiment, L is a nitrogen atom and J and J′ are C₉ polycondensed heterocycloalkyl groups, preferably indoline. Such compound is an analog of Indolinobenzodiazepine (IBDs) dimers.

In a particular embodiment, J and R are selected so as to improve the potency of the drug analog to a compound of formula (I). Accordingly, J and/or J′ can bear an α-β unsaturation relative to L and R and/or R′ may be a C₁-C₃ alkenyl group, optionally substituted by fluorine atoms or a secondary amine; a phenyl group or a methylidene. More particularly, R and/or R′ is a methylidene group.

In a more particular embodiment, m′ and m″ are selected to improve the cytotoxicity of the drug analog to a compound of formula (I). Accordingly, the sum of m′ and m″ can be an even integer, including 2, 4, 6, 8 and 10. In particular, said sum is 2.

In a particular embodiment, a compound of formula (I) is the compound of the formula (III):

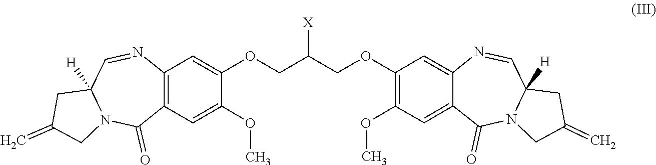

wherein X is as defined above.

In a preferred embodiment, X is an alkynyl group.

Preferably, X is a C₂-C₅ alkynyl.

Even more preferably, X is a prop-2-ynyl group.

Alternatively, X is an azide group.

In the context of the present invention, the terms below have the following definitions unless otherwise mentioned throughout the instant specification:
an alkyl group: a linear or branched saturated hydrocarbon-based aliphatic group comprising, unless otherwise mentioned, from 1 to 6 carbon (noted C₁-C₆ alkyl). By way of examples, mention may be made of, but not limited to: methyl, ethyl, propyl, n-propyl, isopropyl, butyl, isobutyl, sec-butyl, tert-butyl, pentyl, isopentyl, hexyl and isohexyl groups, and the like;
an alkenyl group: a linear or branched hydrocarbon-based aliphatic group bearing at least one carbon-carbon double bond and comprising, unless otherwise mentioned, from 2 to 6 carbon (noted C₂-C₆ alkenyl). Said at least one carbon-carbon double bond can be internal or terminal. By way of examples, mention may be made of, but not limited to: vinyl, allyl, isopropenyl, 1-propenyl, 1-butenyl, 2-butenyl, butadienyl, pentenyl, pentadienyl, hexenyl, and the like.
an alkynyl group: a linear or branched hydrocarbon-based aliphatic group bearing at least one carbon-carbon triple bond and comprising, unless otherwise mentioned, from 2 to 6 carbon (noted C₂-C₆ alkynyl). Said at least one carbon-carbon double bond can be internal or terminal. By way of examples, mention may be made of, but not limited to: ethynyl, &-propynyl, 2-propynyl, 1-butynyl, 2-butynyl, pentynyl, hexynyl and the like.
an heterocycloalkylgroup: a cyclic alkyl group comprising, unless otherwise mentioned, from 3 to 6 carbon atoms and containing 1 or 2 heteroatoms such as oxygen, nitrogen or Sulphur. Such nitrogen atom may be substituted by an oxygen atom in order to form a —N—O bond. Such N—O bond can be in the form of a N-oxide —N⁺—O). Such heterocycloalkyl group may be saturated or partially saturated and unsubstituted or substituted and may be monocyclic or bicyclic;
a carbonyl group: a “C═O” group;
an alkoxy group: an alkyl group as defined herein above, bonded by an ether (—O—) bond. By way of examples, mention may be made of, but not limited to, methoxy, ethoxy, propyloxy, butyloxy, tert-butyloxy, pentyloxy and hexyloxy groups.

The terms used herein with prefixes such as, for example C₁-C₃ or C₁-C₆ encompass hydrocarbon chains that may comprise from 1 to 3 or from 1 to 6 carbon atoms, especially, 1, 2 or 3 or 1, 2, 3, 4, 5 or 6 carbon atoms.

The present invention relates to a composition comprising a compound of formula (I) as provided herein above.

### Kit According to the Invention

The compounds of formula (I) according to the present invention are suitable for forming detectable DNA crosslinks.

Thus, the present invention provides a kit useful for performing labelled DNA crosslinks using compounds of formula (I) according to the invention.

In particular, the present invention provides a kit comprising:
at least a compound of formula (I) as previously defined; and
at least a label bearing a group complementary to the X radical of the compound of formula (I) for a “click-chemistry” reaction, i.e. when the X radical of the compound of formula (I) is an alkyne group, then the complementary group borne by the label is an azide group. Alternatively, when the X radical of the compound of formula (I) is an azide group, then the complementary group borne by the label is an alkyne.

A label comprised in a kit according to the invention can be a moiety that is directly or indirectly detectable. In particular, a label can be selected from dyes, radiolabels and affinity tags.

More particularly, dyes can be selected from the group consisting of fluorescent, luminescent or phosphorescent dyes, preferably dansyl, fluorescein, acridine, rhodamine, coumarine, BODIPY and cyanine dyes.

In a particular embodiment, a kit as provided herein comprises a label that is a fluorescent dye.

A fluorescent dye comprised in a kit according to the invention can be selected among the dyes marketed by Molecular Probes™ such as the Alexa Fluor dyes, Pacific dyes or Texas Red or by other providers for cyanines 3, 5 and 7. In particular, dyes bearing an alkyne or an azide group are commercially available, as examples for Alexa Fluor™ 488, 555, 594 and 647 and for TAMRA (tetramethylrhodamine).

Alternatively, a label comprised in a kit as provided herein can be an affinity tag.

Such label can be, for instance, selected from the group consisting of biotin, His-tag, Flag-tag, strep-tag, sugars, lipids, sterols, PEG-linkers and co-factors. More particularly, said label is a biotinylated label, especially a biotinylated polyethylene glycol label such as Biotin-PEG4 alkyne or other biotins linked to an alkyne or an azide that are commercially available such as Biotin DIBO Alkyne sold by Molecular Probes™.

Finally, a label comprised in a kit as provided herein can be a radiolabel.

Such label can be, for instance, selected from the group consisting of radioactive forms of hydrogen, carbon, phosphorus, Sulphur and iodine. More particularly, said label can be selected from the group consisting of tritium, carbon-11, carbon-14, phosphorus-32, phosphorus-33, Sulphur-33, iodine-123 and iodine-125.

In a particular embodiment, the label is a fluorescent label or a biotin. In a more particular embodiment, the label is a fluorescent label.

A kit as provided herein may further comprise at least one agent selected from the group consisting of copper, more particularly copper(II) precursor with a reducing agent, copper(I) salts or pre-formed copper(I) complexes; a permeabilizing agent; a fixation solution; a washing buffer; and a leaflet comprising explanation for the use of the kit.

In a preferred embodiment, the copper reagent is preferably copper(II) with sodium ascorbate. The permeabilizing agent is a cytoskeleton (CSK) buffer comprising Triton X-100 or any equivalent buffer comprising a detergent suitable for permeabilizing eukaryotic cell membrane. The fixation solution contains paraformaldehyde (PFA) or any equivalent known by the person skilled in the art.

The washing buffer is phosphate-buffered saline (PBS) or any equivalent known by the person skilled in the art.

The present invention also relates to several methods implementing a kit or a compound of the invention.

### Uses and Methods According to The Present Invention

As previously mentioned, a compound of formula (I) of the invention, a composition comprising said compound, or a kit of the invention as defined above, is particularly useful as a research tool.

According to a first aspect, a compound of formula (I) according to the invention enables in vitro or ex vivo detection and visualization of DNA crosslinks in a cell. This labeling allows the localization, quantification or isolation of DNA crosslinks.

Indeed, the labeling allows for the detection of DNA crosslinks in subnuclear regions of the nucleus, thereby allowing to study the localization into the nucleus, and for instance to co-localize with other proteins of interest such as PCNA, RAD18, DNA polymerases, DNA damage response proteins, DNA repair factors and NER/BER/Fanconi crosslinks repair factors or certain genes of interest.

Accordingly, the present invention relates to an in vitro or ex vivo method for visualizing DNA crosslinks generated by compounds of formula (I) according to the invention in a cell, the method comprising:
(a) having at least one cell;
(b) contacting said cell with at least one compound of formula (I) according to the invention under physiological conditions allowing said compound to induce DNA crosslinks in the cell;
(c) contacting the cell obtained in step (b) with at least one label bearing a group which is complementary for a click-chemistry reaction to the X radical of the compound of formula (I) under conditions allowing the reaction of click-chemistry between said X radical and the complementary group;
(d) detecting the label in the cell obtained at step (c).

Said label can in particular be a fluorescent label.

Physiological conditions mean that the considered steps are not carried out in extreme conditions (pH, temperature, pression . . . ) that would lead to a degradation of the cells and/or compounds involved in a method according to the invention.

Step (c) of this method can be performed after or simultaneously to step (b).

In an embodiment where step (b) is performed before step (c), the at least one cell can be incubated with the candidate molecule and/or exposed to the candidate treatment during a period ranging from 1 hour to 5 days, preferably from 1 day to 4 days, for example 3 days.

In particular, the method can comprise a step of cell membrane permeabilization, optionally followed by a step of fixation, between steps (b) and (c). The method can alternatively comprise a step of cell membrane permeabilization, optionally followed by a step of fixation, between steps (c) and (d). According to a particular embodiment, the method comprises a step of cell membrane permeabilization, optionally followed by a step of fixation, between steps (b) and (c) and a step of cell membrane permeabilization, optionally followed by a step of fixation, between steps (c) and (d).

Performing a permeabilization step before the step of fixation allows to improve the quality and the resolution of the labeling. The purpose of using a permeabilization (e.g., CSK or PBS triton 0.5% pre-extraction treatment) prior fixation is to remove soluble proteins and NA loosely bound to chromatin so that the only remaining staining is DNA bound compounds of the invention. By doing so, the resolution of the compounds of the invention cross-linked to DNA is higher due to a lower basal level of fluorescence. This enables the detection of foci targeted by compounds of the invention.

A method of the invention can in particular comprise at least one step of cell membrane permeabilization and at least one step of fixation between steps (b) and (c).

Moreover, the method may comprise at least one step of washing:
between steps (b) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, the method of the invention is such that it comprises:
at least one step of washing between steps (b) and (c); and
at least one step of washing between steps (c) and (d).

More particularly, the method of the invention can be such that it comprises:
between steps (b) and (c), at least one step of washing;
between steps (b) and (c), a step of cell membrane permeabilization followed by a step of fixation; and
between steps (c) and (d), at least one step of washing; and even more particularly:
between steps (b) and (c), and in the following order, a step of washing, a step of cell membrane permeabilization, another step of washing, a step of fixation and another step of washing.

Similarly, the present invention relates to the in vitro or ex vivo use of a compound of formula (I) according to the invention for visualizing DNA crosslinks in a cell, said compound of formula (I) according to the invention being used in combination with a label, preferably a fluorescent label, bearing a group complementary to the X radical of said compounds for a click-chemistry reaction.

The present invention further relates to an in vitro or ex vivo method for assessing the resistance or sensitivity of a tumor in a patient to a crosslinking agent, and in particular to a PBD dimer, more particularly a compound of formula (I) according to the invention, comprising at least the steps of:
(a) having at least one cell from the tumor,
(b) contacting said cell with at least one compound of formula (I) according to the invention under conditions allowing said compound to induce DNA crosslinks in the cell;
(c) contacting the cell obtained in step (b) with at least one label, preferably a fluorescent label, bearing a group which is complementary for a click-chemistry reaction to the X radical of the compounds of formula (I) under conditions allowing the reaction of click-chemistry between the said X radical and the complementary group;
(d) measuring the labelling in the cell obtained at step (c); and
(e) optionally comparing the labeling measured at step (d) to a reference level.

In a particular embodiment, the cell is a cell which is resistant to a crosslinking agent, in particular to a PBD dimer, and more particularly to a compound of formula (I).

The resistance of a cell to a crosslinking agent, in particular to a PBD dimer, and more particularly to a compound of formula (I), refers to the incapacity of the agent to kill the cell, by apoptosis or any other killing process. Accordingly, the resistance of a cell to a crosslinking agent, in particular to a PBD dimer, and more particularly to a compound of formula (I), is then inversely proportionate to the intensity of the label signal.

The reference level can be the intensity measured in a cell known for having a high or low resistance to a crosslinking agent, in particular to a PBD dimer, and more particularly to a compound of formula (I). Preferably, the cell of reference is the closest of the cell to be studied. Alternatively, the reference level can be measured in a cell from the same patient, preferably a non-cancerous cell, for instance a corresponding histological normal reference tissue, in particular from the vicinity of the tumor.

The comparison of the labeling in optional step (e) can be used to determine the resistance or sensitivity of the tumor to a DNA crosslinking agent, in particular to a PBD dimer.

The said PBD dimer in the present method can in particular be a PBD dimer having a formula identical to the one of formula (I) in the absence of radical X, E being a linear alkyl group.

The present invention further relates to an in vitro or ex vivo method for assessing the resistance or sensitivity of a tumor in a patient to a crosslinking agent bearing a X radical as defined above, and in particular to a PBD dimer bearing a X radical as defined above, more particularly to a compound of formula (I) according to the invention and preferably to a compound of formula (II) according to the invention, comprising at least the steps of:
(a) having at least one cell from the tumor,
(b) contacting said cell with at least one said crosslinking agent, in particular a PBD dimer, notably a compound of formula (I) and preferably a compound of formula (II) according to the invention under conditions allowing said compound to induce DNA crosslinks in the cell;
(c) contacting the cell obtained in step (b) with at least one label, preferably a fluorescent label, bearing a group which is complementary for a click-chemistry reaction to the X radical of the crosslinking agent, in particular of the PBD dimer, notably of the compound of formula (I) and preferably of a compound of formula (II) according to the invention under conditions allowing the reaction of click-chemistry between the said X radical and the complementary group;
(d) measuring the labelling in the cell obtained at step (c); and
(e) optionally comparing the labeling measured at step (d) to a reference level.

The resistance of a cell to a crosslinking agent bearing a X radical as defined above, in particular to a PBD dimer bearing a X radical as defined above, more particularly to a compound of formula (I) and preferably to a compound of formula (II) according to the invention, refers to the incapacity of the agent to kill the cell, by apoptosis or any other killing process. Accordingly, the resistance of a cell to a crosslinking agent, in particular to a PBD dimer, more particularly to a compound of formula (I) according to the invention and preferably to a compound of formula (II) according to the invention, is then inversely proportionate to the intensity of the label signal.

The reference level can be the intensity measured in a cell known for having a high or low resistance to a crosslinking agent bearing a X radical as defined above, in particular to a PBD dimer bearing a X radical as defined above, more particularly to a compound of formula (I) according to the invention and preferably to a compound of formula (II) according to the invention. Preferably, the cell of reference is the closest of the cell to be studied. Alternatively, the reference level can be measured in a cell from the same patient, preferably a non-cancerous cell, for instance a corresponding histological normal reference tissue, in particular from the vicinity of the tumor.

The comparison of the labeling in optional step (e) can be used to determine the resistance or sensitivity of the tumor to a DNA crosslinking agent bearing a X radical as defined above, in particular to a PBD dimer bearing a X radical as defined above.

The said PBD dimer in the present method can in particular be a PBD dimer having a formula identical to the one of formula (I) or to the one of formula (II) in the absence of radical X.

As defined herein above, a condition allowing a click-chemistry reaction, in particular a CuAAC is the presence of copper. More particularly in said CuAAC, copper(I) act as a catalyst and can be provided by the use of copper(II) precursors with a reducing agent, such as sodium ascorbate or p-hydroquinone for instance, by copper(I) salts or by pre-formed copper(I) complexes.

In particular, the method can comprise a step of cell membrane permeabilization, optionally followed by a step of fixation, between steps (b) and (c). The method can alternatively comprise a step of cell membrane permeabilization, optionally followed by a step of fixation, between steps (c) and (d). According to a particular embodiment, the method comprises a step of cell membrane permeabilization, optionally followed by a step of fixation, between steps (b) and (c) and a step of cell membrane permeabilization, optionally followed by a step of fixation, between steps (c) and (d).

A method of the invention can in particular comprise at least one step of cell membrane permeabilization and at least one step of fixation between steps (b) and (c).

Moreover, the method may comprise at least one step of washing:
between steps (b) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, the method of the invention is such that it comprises:
at least one step of washing between steps (b) and (c); and
at least one step of washing between steps (c) and (d).

More particularly, the method of the invention can be such that it comprises:
between steps (b) and (c), at least one step of washing;
between steps (b) and (c), a step of cell membrane permeabilization followed by a step of fixation; and
between steps (c) and (d), at least one step of washing; and even more particularly:
between steps (b) and (c), and in the following order, a step of washing, a step of cell membrane permeabilization, another step of washing, a step of fixation and another step of washing.

In a particular embodiment, a resistance to a crosslinking agent, in particular to a PBD dimer, and more particularly to a compound of formula (I), can be determined based on a change of localization of the labeling.

Indeed, the impact of the candidate molecule and/or candidate treatment on the localization of the labeling can be considered as a marker of the resistance of the cell to a crosslinking agent, in particular to a PBD dimer, and more particularly to a compound of formula (I). Therefore, the impact of the candidate molecule and/or candidate treatment on the morphology of foci can also be studied.

In a particular embodiment, a resistance to a crosslinking agent bearing a X radical as defined above, in particular to a PBD dimer bearing a X radical as defined above, more particularly to a compound of formula (I) more particularly to a compound of formula (I) and preferably to a compound of formula (II) according to the invention, can be determined based on a change of localization of the labeling.

Indeed, the impact of the candidate molecule and/or candidate treatment on the localization of the labeling can be considered as a marker of the resistance of the cell to a crosslinking agent bearing a X radical as defined above, in particular to a PBD dimer bearing a X radical as defined above, more particularly to a compound of formula (I) according to the invention and preferably to a compound of formula (II) according to the invention. Therefore, the impact of the candidate molecule and/or candidate treatment on the morphology of foci can also be studied.

The present invention also pertains to an in vitro or ex vivo method for identifying or screening a candidate molecule and/or a candidate treatment for its ability to improve the interstrand and/or intrastrand DNA crosslinking induced by a crosslinking agent, in particular by a PBD dimer. Such method is in particular useful to prevent, overcome or reduce the resistance of cells, in particular of tumor cells, to crosslinking agents, and in particular to PBD dimers.

The present invention accordingly pertains to an in vitro or ex vivo method for identifying or screening a candidate molecule and/or a candidate treatment for its ability to improve the efficiency of a crosslinking agent, in particular of a PBD dimer, and more particularly of a PBD dimer of formula (I) of the invention, the method comprising:
(a) having at least one cell, in particular a tumor cell;
(b) contacting said cell with the candidate molecule and/or applying the candidate treatment to said cell;
(b′) contacting the cell with at least one compound of formula (I) according to the invention under conditions allowing said compound to induce DNA crosslinks in the cell;
(c) contacting the cell obtained after the previous steps with at least one label, preferably a fluorescent label, bearing an group which is complementary to the X radical of the compound according to the invention, under conditions allowing the reaction of click-chemistry between the X radical and the complementary group;
(d) measuring the labelling in the cell obtained at step (c); and
(e) comparing the intensity of the labelling obtained at step (d) to a reference labelling intensity obtained when the method is performed in the absence of the candidate molecule and/or of the candidate treatment;
wherein said steps (b) and (b′) can occur simultaneously or sequentially, preferably in that order.

The candidate molecule and/or candidate treatment can be considered as improving the interstrand or intrastrand DNA crosslinking induced by a crosslinking agent, in particular by a PBD dimer, more particularly by a PBD dimer of the invention, if the intensity of the labelling is increased in the presence of the candidate molecule and/or candidate treatment when compared to the intensity of the labelling in the absence of the said candidate.

The present invention accordingly pertains to an in vitro or ex vivo method for identifying or screening a candidate molecule and/or a candidate treatment for its ability to improve the efficiency of a crosslinking agent bearing a X radical as defined above, in particular of a PBD dimer bearing a X radical as defined above, more particularly of a PBD dimer of formula (I) of the invention and preferably of a compound of formula (II) of the invention, the method comprising:
(a) having at least one cell, in particular a tumor cell;
(b) contacting said cell with the candidate molecule and/or applying the candidate treatment to said cell;
(b′) contacting the cell with at least one crosslinking agent, in particular a PBD dimer, notably a compound of formula (I) according to the invention and preferably a compound of formula (II) according to the invention under conditions allowing said compound to induce DNA crosslinks in the cell;
(c) contacting the cell obtained after the previous steps with at least one label, preferably a fluorescent label, bearing an group which is complementary to the X radical of the said crosslinking agent, in particular a PBD dimer, notably a compound of formula (I) compound according to the invention and preferably a compound of formula (II) according to the invention, under conditions allowing the reaction of click-chemistry between the X radical and the complementary group;
(d) measuring the labelling in the cell obtained at step (c); and
(e) comparing the intensity of the labelling obtained at step (d) to a reference labelling intensity obtained when the method is performed in the absence of the candidate molecule and/or of the candidate treatment;
wherein said steps (b) and (b′) can occur simultaneously or sequentially, preferably in that order.

The candidate molecule and/or candidate treatment can be considered as improving the interstrand or intrastrand DNA crosslinking induced by a crosslinking agent bearing a X radical as defined above, in particular by a PBD dimer bearing a X radical as defined above, more particularly by a PBD dimer of the invention, if the intensity of the labelling is increased in the presence of the candidate molecule and/or candidate treatment when compared to the intensity of the labelling in the absence of the said candidate.

In particular, the method can comprise a step of cell membrane permeabilization, optionally followed by a step of fixation, between steps (b) and (c). The method can alternatively comprise a step of cell membrane permeabilization, optionally followed by a step of fixation, between steps (c) and (d). According to a particular embodiment, the method comprises a step of cell membrane permeabilization, optionally followed by a step of fixation, between steps (b) and (c) and a step of cell membrane permeabilization, optionally followed by a step of fixation, between steps (c) and (d).

In a particular embodiment, the method comprises, or further comprises, a step of cell membrane permeabilization, optionally followed by a step of fixation, between steps (b) and (b′).

A method of the invention can in particular comprise at least one step of cell membrane permeabilization and at least one step of fixation between steps (b) and (c).

Moreover, the method may comprise at least one step of washing:
between steps (b) and (b′), in order to remove the candidate molecule; and/or
between steps (b′) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, there is no washing step between steps (b) and (b′) when they are performed sequentially.

More particularly, the method may comprise at least one step of washing:
between steps (b′) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, the method of the invention is such that it comprises:
at least one step of washing between steps (b) and (c); and
at least one step of washing between steps (c) and (d).

More particularly, the method of the invention can be such that it comprises:
between steps (b) and (c), at least one step of washing;
between steps (b) and (c), a step of cell membrane permeabilization followed by a step of fixation; and
between steps (c) and (d), at least one step of washing; and even more particularly:
between steps (b) and (c), and in the following order, a step of washing, a step of cell membrane permeabilization, another step of washing, a step of fixation and another step of washing.

In a particular embodiment, the cells used in the methods of the present invention are cancer cells. It can stem from a cancer cell line or a cell from primary tumors. It can be resistant to a crosslinking agent, and in particular to a PBD dimer, more particularly a compound of formula (I) according to the invention. Preferably the cells are mammalian cells, and more specifically human cells.

In all the methods of the invention, the labeling of DNA crosslinks authorizes the quantification of the number of DNA crosslinks generated by a compounds of formula (I) of the invention. For example, if the label is fluorescent, the amount of fluorescence can be measured, this amount being proportional to the number of DNA crosslinks generated by a compounds of formula (I) of the invention. If the label is radioactive, then the amount of radioactivity is measured.

All the methods of the invention may comprise at least one step of washing. The said at least one step of washing in a method of the invention may more particularly be performed after contacting a cell with a compound according to the invention and/or be performed after contacting a cell with a label as disclosed herein.

All the methods of the invention may comprise at least one step of cell membrane permeabilization. The said at least one step of cell membrane permeabilization (also termed cell permeabilization) in a method of the invention may more particularly be performed after contacting a cell with a compound according to the invention. The at least one step of permeabilization in a method of the invention can moreover be followed by a step of fixation, i.e. a step of fixation can be performed consecutively after the said at least one step of permeabilization.

In a particular embodiment, a method of the invention comprises at least one step of washing and at least one step of cell membrane permeabilization, the at least one step of cell membrane permeabilization being optionally followed by a step of fixation.

Finally, the present invention also pertains to the use of a compound or of a kit as provided herein for isolating DNA crosslinks generated by compounds of formula (I) according to the invention, more specifically isolating sequences comprising said DNA crosslinks by a pull-down methodology as the present invention authorizes the high throughput sequencing of the isolated sequences.

The present invention accordingly pertains to a method comprising:
(a) providing at least one cell;
(b) contacting said at least one cell with a compound of formula (I) according to the invention under conditions allowing said compound to induce DNA crosslinks in the cell;
(c) purifying or isolating the genomic DNA from the at least one cell obtained from step (b),
(d) adding an affinity tag bearing a complementary group to the radical X of a compound of formula (I) for a click-chemistry reaction under conditions allowing the reaction of click-chemistry between the said X radical and the complementary group; and
(e) isolating or purifying the genomic DNA linked to the affinity tag.

Optionally, the method may comprise a step of removing RNA, particularly during step (c).

Optionally, the method may comprise a step of fragmenting DNA before step (e).

In a particular embodiment, step (e) is carried out by contacting the DNA with a solid support on which a molecule able to bind the affinity tag has been immobilized such as beads.

In a particular embodiment, the method comprises an additional step of reversing the DNA crosslinks after step (e), for example by using thiourea.

In a particular embodiment, the affinity tag is a biotin. Biotins linked to alkynes or azides are commercially available (Biotin-PEG4 alkyne and Azide-PEG3-biotin by Sigma Aldrich). Then streptavidin can be used in step (e) for isolating or purifying the genomic DNA linked to the biotin. This method can be easily adapted with another couple of affinity tag-binding agent.

The recovered DNA can be used by the person skilled in the art for any kind of analysis. In particular, this recovered DNA can be sequenced.

### Process for the Preparation of Compounds According to the Invention

The present invention also pertains to processes for the preparation of the compounds herein provided and their precursors, as illustrated in the examples. The present invention also provides novel intermediates for use in such processes.

More particularly, the present invention provides a process for the preparation of compounds of the formula (II):

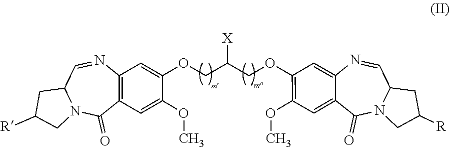

wherein:
m′ and m″ are integers independently ranging from 1 to 10 and their sum is less than 12,
X, R and R′ are as defined above.

In a particular embodiment, and as illustrated in the following scheme, the pyrrolidine derivative of formula (A) is coupled with the PBD dimer core of formula (B) to form the bis-nitroamide of formula (C). A subsequent ring cyclization provides the bis-lactam of formula (D). The amide nitrogen of said bis-lactam is then protected to give the compound of formula (E) which may then be converted to a PBDs analog of formula (II) passing through the bis-aminol of formula (F).

This synthesis process of a compound of formula (II) according to the invention implements a convergent approach, in a process easy to implement and which yield a pure form.

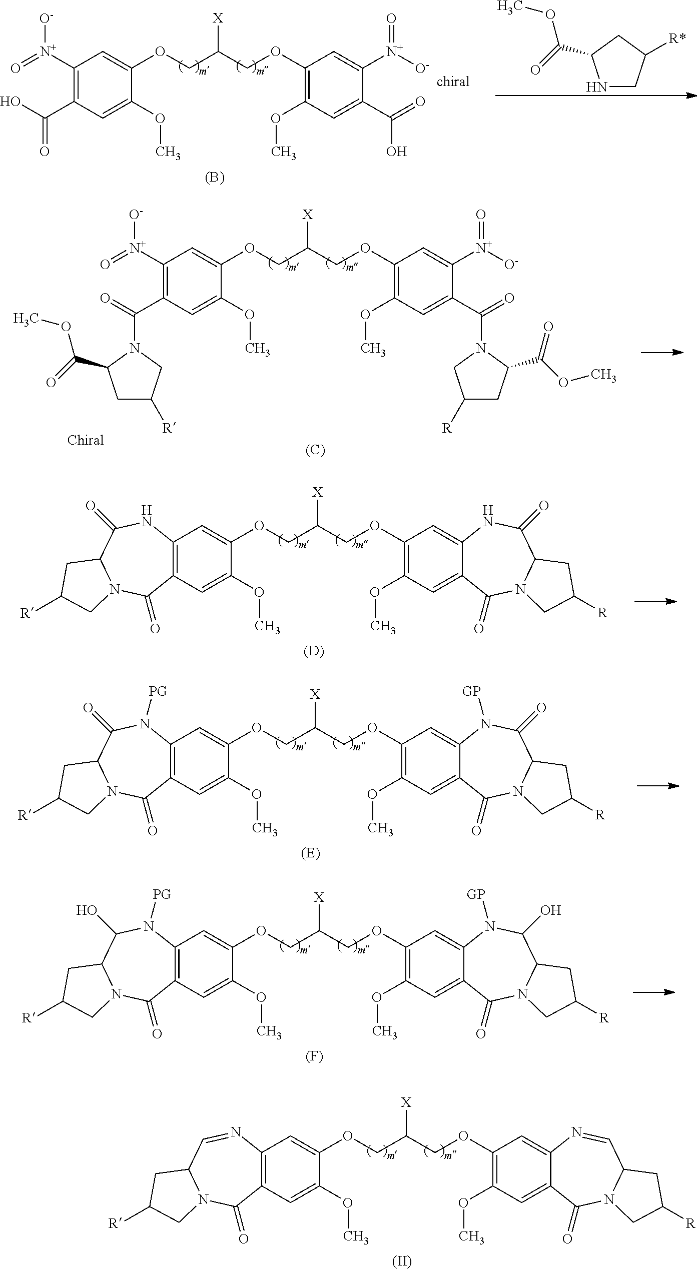

Alternatively, it is possible to synthesize a compound of the invention by a reduction of the bis-nitroamide (C) to obtain the corresponding bis-nitroaldehyde (C′). A subsequent reductive cyclization by an appropriate reducing agent affords to obtain the compound of formula (II) according to the following Scheme:

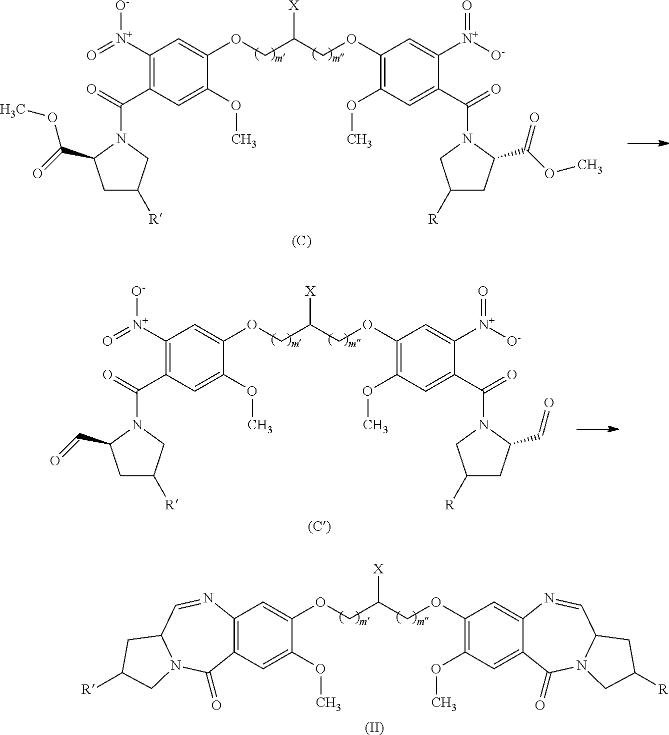

In another alternative embodiment, a compound of the invention may be prepared using a linear approach implementing a mono PBD unit (V) by dialkylation according to the following scheme:

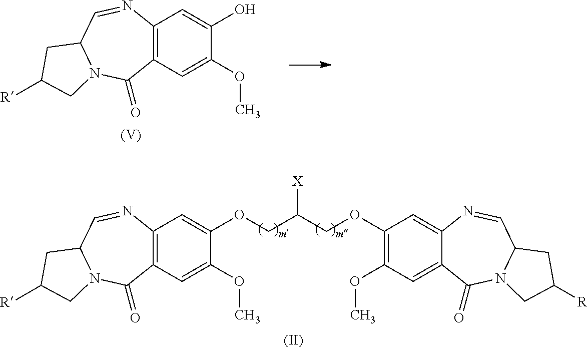

In a particular embodiment, the present invention pertains to a process for the preparation of a compound of the formula (III):

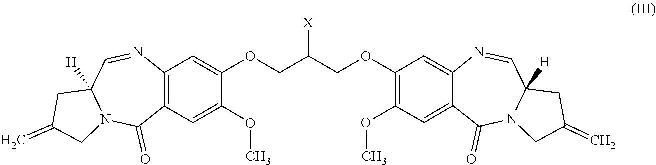

wherein X is as defined above.

In another of its aspects, the invention also provides the compounds as defined below wherein m′, m″, R, R′ and X are as defined above, which are useful as intermediates in the synthesis of compounds of formula of the invention as defined above:

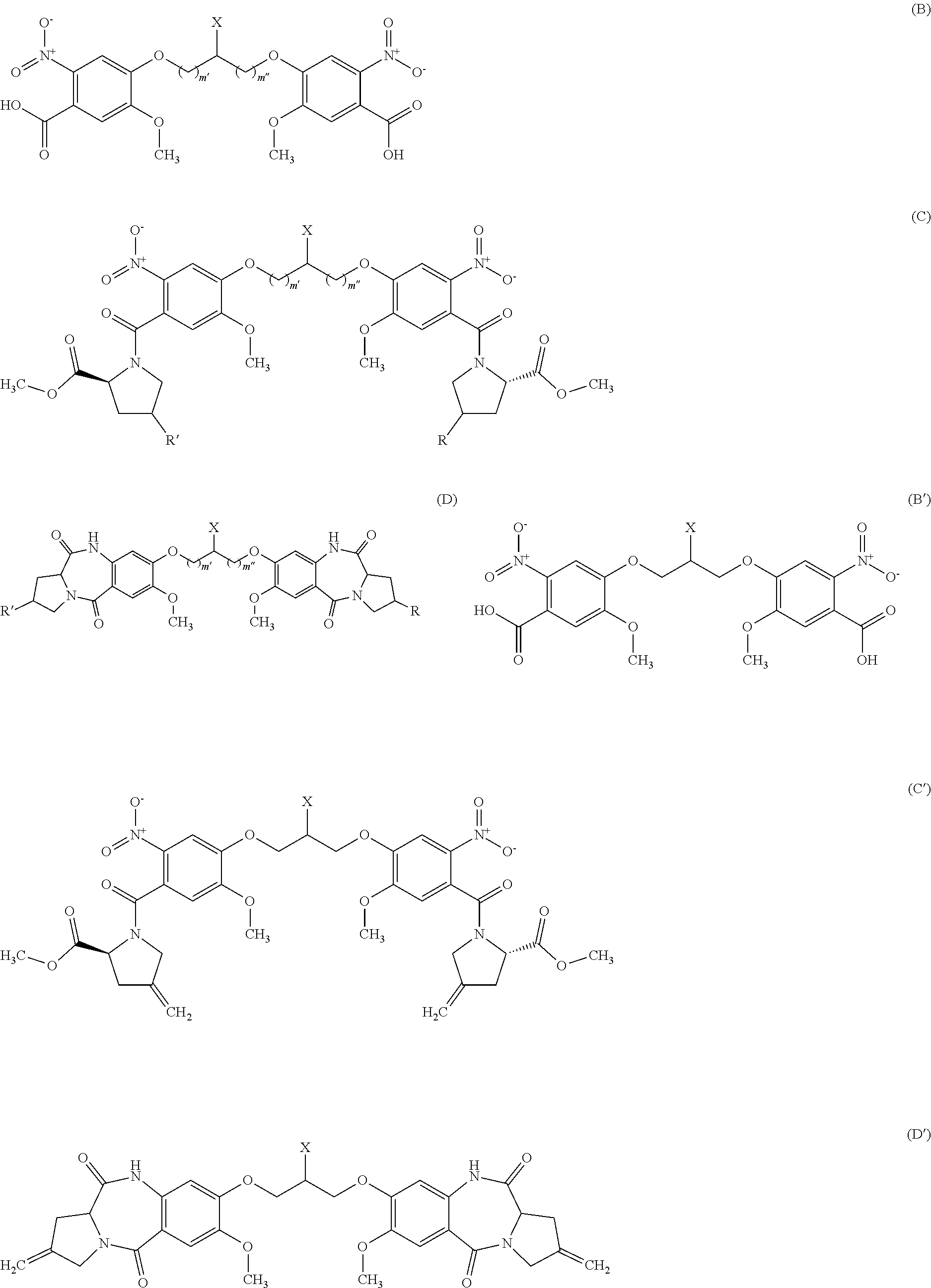

Some particular embodiments of the compounds according to the invention are described, with their names, methods of preparation and analytical data, in the following examples.

Commercially available reagents and solvents were used without additional purification. The petroleum ether refers to the fraction with distillation range 40-70° C. Thin layer chromatography (TLC) was performed on precoated aluminum sheets of silica (60 F254 nm, Merck) and visualized using short-wave UV light. Flash column chromatography was carried out on Macherey-Nagel silica gel 60 (size 70-230 mesh). Column chromatography was also performed on a Reveleris purification system using Reveleris Flash silica cartridges or C18 40 μM cartridges. Semipreparative HPLC was performed on an Agilent Infinity system, flow 3 mL/min, mobile phase was a mixture of water and methanol, both containing trifluoroacetic acid at 0.1%. ¹H and ¹³C NMR spectra were recorded at room temperature on Bruker AC400, AC300 or AC250 spectrometers. Chemical shifts (δ) are reported in parts per million (ppm) with internal reference and coupling values (J) in hertz.

Abbreviations for peaks are, s: singlet, d: doublet, t: triplet, q: quadruplet, quint: quintuplet, sex: sextuplet and m: multiplet. The spectra recorded are consistent with the proposed structures. Reaction monitoring and purity of compounds were recorded by using analytical Agilent Infinity high performance liquid chromatography (Column Zorbax SB-C18 1.8 μM (2.1×50 mm); Mobile phase (A: 0.1% FA H20, B: 0.1% FA MeCN, Time/%B 0/10, 4/90, 7/90, 9/10, 10/10); Flow rate 0.3 mL/min; Diluent MeOH) with DAD at 254 nM. Low-resolution mass spectra were obtained with Agilent SQ G6120B mass spectrometer in positive electrospray mode. High-resolution mass spectra were performed at the Spectropole of Aix-Marseille University, Marseille, France. All tested compounds yielded data consistent with a purity of ≥95%.

The present invention is further illustrated by, without in any way being limited to, the examples herein.

### EXAMPLES

### Example 1. Preparation of Compounds (2-6)

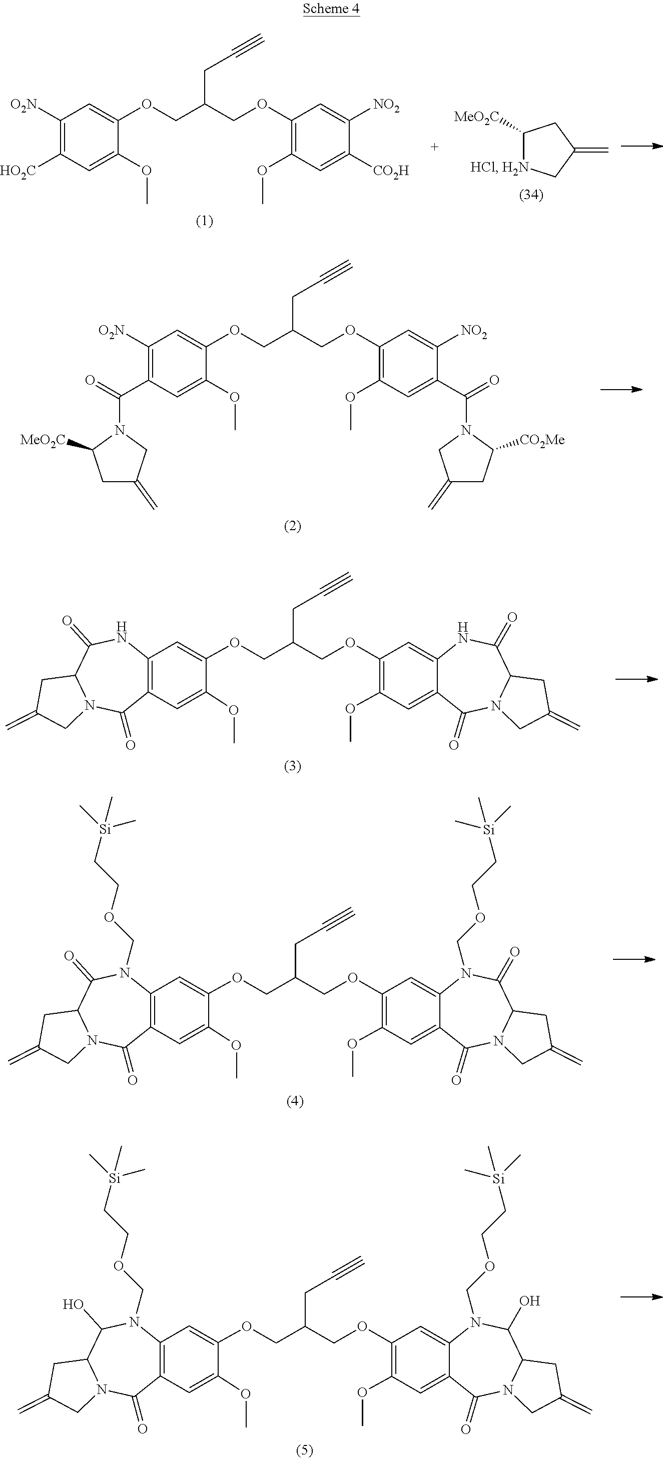

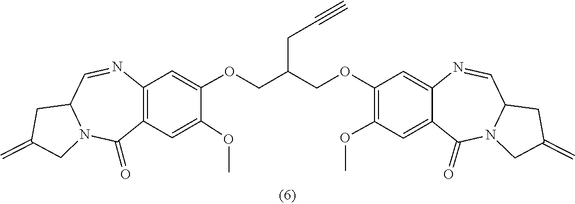

### 1,1′-(4,4′-((2-(prop-2-yn-1-yl)propane-1,3-diyl)bis(oxy))bis(5-methoxy-2-nitrobenzoyl))bis[(2S)-4-methylenepyrrolidine-2-carboxylic acid methyl ester] (2)

Under argon, to a solution of 4,4′-((2-(prop-2-yn-1-yl)propane-1,3-diyl)bis(oxy))bis(5-methoxy-2-nitrobenzoic acid) (1) (699 mg, 1.39 mmol) in dichloromethane (5 mL), was successively added dimethylformamide (2 drops) and oxalyl chloride (0.36 mL, 4.17 mmol). The reaction mixture was stirred at room temperature for 2 hours and concentrated under reduced pressure. The corresponding acid chloride was suspended in dichloromethane (3 mL) and the solution was added dropwise, at 0° C., to a mixture of (S)-4-methylenepyrrolidine-2-carboxylic acid methyl ester hydrochloride (34) (493 mg, 2.78 mmol) and triethylamine (0.854 mL, 6.12 mmol) in dichloromethane (5 mL). After addition, the resulting mixture was stirred at 0° C. for additional 15 minutes, then, successively diluted with CH₂Cl₂. washed with aqueous 1N HCl solution (3×30 mL), saturated aqueous Na₂CO₃ (30 mL) solution, and dried over Na₂SO₄. The solvent was distillated off under reduced pressure and the residue was purified by column chromatographygradient petroleum ether-EtOAc (3:7 to 0:1) to afford 1,1′-(4,4′-((2-(prop-2-yn-1-yl)propane-1,3-diyl)bis(oxy))bis(5-methoxy-2-nitrobenzoyl))bis[(2S)-4-methylenepyrrolidine-2-carboxylic acid methyl ester] (2) (960 mg, 92%) as a yellow powder. ¹H NMR (250 MHz, CDCl₃) rotamers; ¹³C NMR (63 MHZ, CDCl₃) rotamers; LCMS C₃₆H₃₈N₄O₁₄ Rt=6.873 min, ESI⁺ m/z=751.0 (M+H).

### 8,8′-((2-(prop-2-yn-1-yl)propane-1,3-diyl)bis(oxy))bis(7-methoxy-2-methylene-2,3-dihydro-1H-benzo[e]pyrrolo[1,2-a][1,4]diazepine-5,11(10H,11aH)-dione) (3)

Under argon, at room temperature, to a suspension of (2) (750 mg, 1.0 mmol) and zinc (825 mg, 12.6 mmol) in anhydrous tetrahydrofuran (10.5 mL), was successively injected anhydrous methanol (10.5 mL) and glacial acetic acid (0.66 mL). The reaction mixture was stirred at 45° C. for 4 hours, filtrated through cotton, and the filtrate was concentrated under reduced pressure. The residue was purified by column chromatography, on Reveleris system, eluent CHCl₃—MeOH (99:1) to afford 8,8′-((2-(prop-2-yn-1-yl)propane-1,3-diyl)bis(oxy))bis(7-methoxy-2-methylene-2.3-dihydro-1H-benzo[e]pyrrolo[1,2-a][1,4]diazepine-5,11(10H, 11aH)-dione) (3) (620 mg., 99%) as a yellow powder. ¹H NMR (250 MHZ, CDCl₃)) δ8.95 (s, 2H), 7.43 (s, 2H), 6.57 (s, 2H), 5.17 (broad s, 2H), 5.11 (broad s, 2H),4.42 (d, J=16.0 Hz, 2H), 4.21 -4.16 (m, 8H), 3.89 (s, 6H), 3.43 (d, J=15.5 Hz, 2H), 2.89-2.79 (3, 2H), 2.71 (sept, J=5.7 Hz, 1H), 2.55 (dd, J=7.4 and 2.6 Hz, 2H) and 2.06 (t, J=2.6 Hz, 1H); ¹³C NMR (63 MHZ, CDCl₃); δ170.6, 165.3, 151.6, 146.9, 141.4, 129.8, 119.2, 112.7, 109.0, 105.8, 81.0, 70.9, 68.2, 56.9, 56.3, 51.5, 37.7, 31.8, 18.0; LCMS C₃₄H₃N₄O₈ Rt=5.832 min, ESI⁺ m/z=627.0 (M+H).

### 8,8′-((2-(prop-2-yn-1-yl)-propane-1,3-diyl)bis(oxy))bis(7-methoxy-2-methylene-10-((2-(trimethylsilyl)ethoxy)methyl)-2,3-dihydro-1H-benzo[e]pyrrolo[1,2-a][1,4]diazepine-5,11(10H,11aH)-dione) (4)

Under argon, at −50° C., to a solution of (3) (590 mg, 0.94 mmol) in a mixture of tetrahydrofuran-dimethylformamide (4:2, 60 mL), was added 60% sodium hydride in oil (120 mg, 3.01 mmol). The suspension was stirred at room temperature for 1 hr, then coolded to −78° C. (2-(Chloromethoxy)ethyl)trimethylsilane (0.78 mL, 4.30 mmol) was added dropwise and resulting mixture was stirred for 1 hr, before being allowed to warm to room temperature overnight. The solvent was distillated off under reduced pressure and the residue was purified by column chromatography, gradient petroleum ether-EtOAc (1:0 to 0:1) to afford 8,8′-((2-(prop-2-yn-1-yl)propane-1,3-diyl)bis(oxy))bis(7-methoxy-2-methylene-10-((2- (trimethylsilyl)ethoxy)methyl)-2.3-dihydro-1H-benzo[e]pyrrolo[1.2-a][1,4]diazepine-5,11(10H,11aH)-dione (4) (659 mg, 79%) as light yellow powder. ¹H NMR (250 MHZ, CDCl₃) δ7.31 (s, 2H), 7.22 (2×s, 2H), 5.46 (d, J=10.0 Hz, 2H), 5.16 (broad s, 2H), 5.09 (broad s, 2H), 4.70 (d, J=10.0 Hz, 2H), 4.36-4.16 (m, 10H), 3.85 (s, 6H), 3.81-3.59 (m, 4H), 3.40 (d, J=17.2 Hz, 2H), 2.87-2.66 (m, 3H), 2.60 (dd, J=7.9 and 2.3 Hz, 2H), 1.99 (t, J=2.3 Hz, 1H), 0.94 (2×t, J=8.3 Hz, 4H) and 0.02 (2×s, 18H); ¹³C NMR (63 MHz, CDCl₃) δ169.4, 164.7, 150.9, 147.3, 141.6, 133.5, 121.8, 111.2, 108.5, 107.0, 80.7, 70.4, 67.8, 66.7, 60.0, 57.2, 55.8, 50.6, 37.6, 32.0, 17.9, 17.6 and −1.60; LCMS C₄₆H₆₂N₄O₁₀Si₂ Rt=8.543 min.

### 8,8′-((2-(prop-2-yn-1-yl)propane-1,3-diyl)bis(oxy))bis(7-methoxy-2-methylene-2,3-dihydro-1H-benzo[e]pyrrolo[1,2-a][1,4]diazepin-5(11aH)-one) (6)

Under argon, at −78° C., to a solution of (4) (248 mg, 0.28 mmol) in tetrahydrofuran (10 mL) was added dropwise 1M superhydride solution in tetrahydrofuran (1.67 mL, 1.67 mmol). The resulting mixture was stirred at −78° C. for 45 min and subsequently quenched by addition of a mixture of MeCN-H₂O (1:1.2.4 mL). The solvent was distillated off under reduced pressure and the residue was dissolved in DMSO before purification by column chromatography onto C18, gradient H₂O-MeCN (95:5 to 5:95, each containing 0.05% of AcOH) to afford 8,8′-((2-(prop-2-yn-1-yl)propane-1,3-diyl)bis(oxy))bis(7-methoxy-2-methylene-2,3-dihydro-1H- benzo[e]pyrrolo[1,2-a][1,4]diazepin-5(11aH)-one) (6) (108 mg, 65%) as light yellow powder [α]²⁰_{D}=+304 (c=0.117, CHCl₃). ¹H NMR (300 MHz, CDCl₃) δ7.66 (d, J=4.2 Hz, 2H), 7.47 (s, 2H), 6.87 (s, 2H), 5.18 (broad s, 2H), 5.15 (broad s, 2H), 4.30-4.15 (m, 8H), 3.89 (s, 6H), 3.84-3.77 (m, 2H), 3.10 (dd, J=15.6 and 9.2 Hz, 2H), 2.92 (d, J=15.6 Hz, 2H), 2.75-2.65 (m, 1H), 2.61 (dd, J=6.1 and 2.4 Hz, 2H), 2.00 (d, J=2.4 Hz, 1H); NMR (75 MHz, CDCl₃) 8 164.9, 162.6, 150.9, 148.2, 141.7, 140.7, 120.2, 111.8, 111.1, 109.6, 81.2, 70.7, 68.2, 56.2, 53.9, 51.5, 37.8, 35.6, 18.1; LCMS C₃₄H₃₄N₄O₆ Rt=5.697 min, ESI⁺ m/z=595.3 (M+H); HRMS [M+H] Calcd for C₃₄H₃₄N₄O₆ m/z 595.2551, found 595.2551.

### Example 2. Preparation of Compounds (6) and (7)

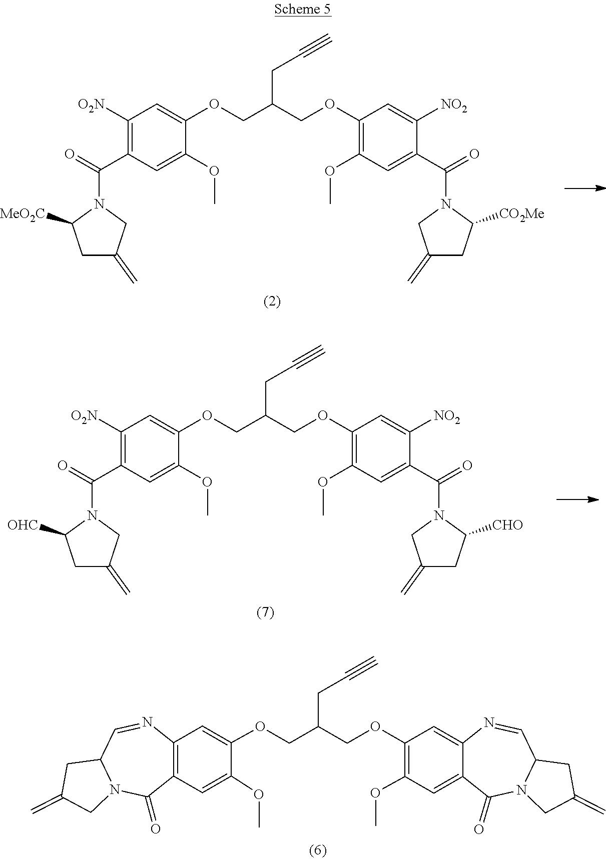

### 1,1′-(4,4′-((2-(prop-2-yn-1-yl)propane-1,3-diyl)bis(oxy))bis(5-methoxy-2-nitrobenzoyl))bis[(2S)-4-methylenepyrrolidine-2-formaldehyde] (7)

Under argon, at −78° C., to a solution of (2) (450 mg, 0.6 mmol) in dichloromethane (15 mL), was added dropwise 1N diisobutylaluminium hydride solution in hexane (4.2 mL, 4.2 mmol). After addition, the reaction mixture was stirred at −78° C. for 15 min and quenched by addition of MeOH (10 mL). The mixture was stirred for an additional 15 min and then, an aqueous solution of 1N HCI (15 mL) was added and the resulting solution was allowed to stir at −78° C. for an additional 15 min. The aqueous layer was extracted with CH₂Cl₂ (2×30 mL) and combined. Organic layers were washed with saturated aqueous Na₂CO₃ (2×20 mL) then dried over Na₂SO₄. The solvent was distillated off under reduced pressure and the residue was purified by column chromatography, eluent CH₂C₁₂—MeOH (95:5) to afford 1,1′-(4,4′-((2-(prop-2-yn-1-yl)propane-1,3-diyl)bis(oxy))bis(5-methoxy-2-nitrobenzoyl))bis[(2S)-4- methylenepyrrolidine-2-formaldehyde] (7) (370 mg, 89%) as a light yellow powder. ¹H NMR (250 MHz, CDCl₃) rotamers; ¹³C NMR (63 MHZ, CDCl₃) rotamers; LCMS C₃₄H₃₄N₄O₁₂ Rt=6.214 min, ESI⁺ m/z=691.0 (M+H).

### 8,8′-((2-(Prop-2-yn-1-yl)propane-1,3-diyl)bis(oxy))bis(7-methoxy-2-methylene-2,3-dihydro-1H-benzo[e]pyrrolo[1,2-a][1,4]diazepin-5(11aH)-one) (6)

Method (A). Under argon, at room temperature, to a suspension of (7) (50 mg, 0.07 mmol) and zinc (70 mg, 0.80 mmol) in anhydrous tetrahydrofuran (1 mL), was successively injected anhydrous methanol (1 mL) and glacial acetic acid (60 μL). The reaction mixture was stirred at 40° C. for 15 min, filtrated, and the filtrate was concentrated under reduced pressure. The residue was purified by column chromatography. gradient CH₂Cl₂—MeOH (10:0.5 to 10:1) to afford 8,8′-((2-(Prop-2-yn-1-yl)propane-1,3-diyl)bis(oxy))bis(7-methoxy-2-methylene-2,3-dihydro-1H-benzo[e]pyrrolo[1,2-a][1,4]diazepin-5(11aH)-one) (6) as a yellow powder.

Method (B). At room temperature, to a solution of (7) (71 mg, 0.10 mmol) in a mixture of tetrahydrofuran-water (5:3, 8 mL) was added portionwise sodium hydrosulfite (286 mg, 1.64 mmol) over 2 min. The resulting mixture was stirred for 1.2 hours, and concentrated under reduced pressure. The solid residue was suspended in anhydrous MeOH (10 mL), and AcCl (0.14 mL, 2.00 mmol) was added dropwise. The solution was stirred overnight, concentrated under reduced pressure and the residue was purified by column chromatography onto C18, gradient H2O—MeCN (95:5 to 5:95, each containing 0.05% of AcOH) to afford 8.8′-((2-(Prop-2-yn-1-yl)propane-1,3-diyl)bis(oxy))bis(7-methoxy-2-methylene-2,3-dihydro-1H-benzo[e]pyrrolo[1,2-a][1,4]diazepin-5(11aH)-one) (6) as a yellow powder.

### Example 3. Preparation of Compounds (6)

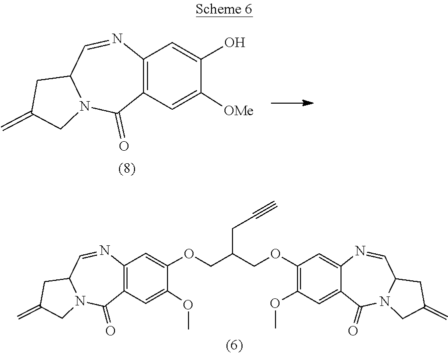

### 8,8′-((2-(Prop-2-yn-1-yl)propane-1,3-diyl)bis(oxy))bis(7-methoxy-2-methylene-2,3-dihydro-1H-benzo[e]pyrrolo[1,2-a][1,4]diazepin-5(11aH)-one) (6)

Under argon, at 0° C., to a solution of (11aS)-8-Hydroxy-7-methoxy-2-methylene-1,2,3,11a-tetrahydro-5H-pyrrolo[2,1-c][1,4]benzodiazepin-5-one (8) (258 mg, 1 mmol) and triphenylphosphine (447 mg, 1.5 mmol) in anhydrous tetrahydrofuran (5 mL) was added dropwise diethyl azodicarboxylate (0.24 mL, 2 mmol) over 5 min. After addition, the resulting mixture was vigorously stirred at 0° C. for 30 min and a solution of 2-(prop-2-ynyl)propane-1,3-diol (37) (35 mg, 0.5 mmol) in anhydrous tetrahydrofuran (2 mL) was added dropwise. After addition, stirring was maintained at 0° C. for an additional 1 hour. The reaction mixture was concentrated under reduced pressure and the crude residue was purified by column chromatography, gradient CH₂Cl₂—MeOH (10:0.5 to 10:1) to afford 8,8′-((2-(prop-2-yn-1-yl)propane-1,3-diyl)bis(oxy))bis(7-methoxy-2-methylene-2,3-dihydro-1H-benzo[e]pyrrolo[1,2-a][1,4]diazepin-5 (11aH)-one) (6) as a yellow powder.

### Example 4. Preparation of Compound (10-16)

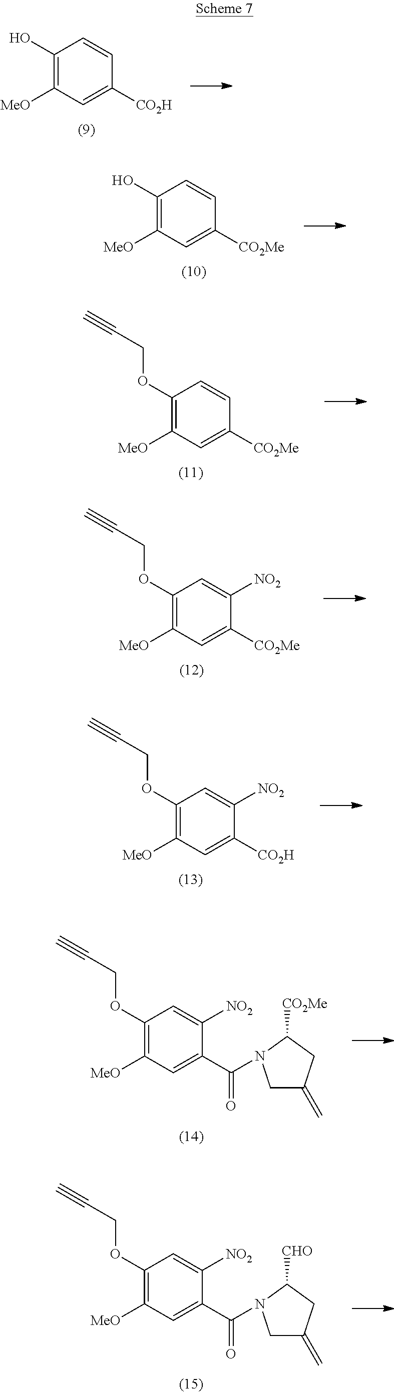

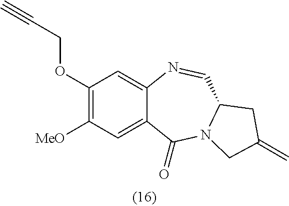 [SMILES: C#CCOc1cc2c(cc1OC)C(=O)N1CC(=C)C[C@H]1C=N2]

### 4-Hydroxy-3-methoxybenzoic acid methyl ester (10)

Under argon, at room temperature, to a suspension of isovanillic acid (10.0 g, 59 mmol) in absolute methanol (100 mL) was added dropwise thionyl chloride (3.9 mL, 53.1 mmol). The resulting mixture was stirred at room temperature for 72 hours then concentrated under reduced pressure. The solid residue was poured into cold H₂O (40 mL) and basified, under stirring, with saturated aqueous solution of NaHCO₃ until pH7-8. The remaining precipitate was collected by filtration affording 4-hydroxy-3-methoxybenzoic acid methyl ester (10) (10.8 g, quantitative) as a light brown solid. ¹H NMR (250 MHz, CDCl₃) δ7.63 (dd, J=8.3 and 2.0 Hz, 1H), 7.54 (d, J=2.0 Hz, 1H), 6.93 (d, J=8.3 Hz, 1H), 3.92 (s, 3H) and 3.88 (s, 3H); LCMS C₉H₁₀O₄ Rt=5.248 min, ESI⁺ m/z=182.9 (M+H).

### 3-Methoxy-4-(prop-2-yn-1-yloxy)benzoic acid methyl ester (11)

Under argon, to a suspension of (10) (1.82 g, 10 mmol) and potassium carbonate (4.88 g, 40 mmol) in acetone (30 mL), was injected propargyl bromide (2.6 mL, 30 mmol). The resulting mixture was refluxed overnight and directly filtrated without any work up. The filtrate was concentrated under reduced pressure and the residue was crystallized from MeOH (20 mL) to afford 3-methoxy-4-(prop-2-yn-1-yloxy)benzoic acid methyl ester (11) (2.0 g, 91%) as a light brown plates. ¹H NMR (300 MHz, CDCl₃) δ7.66 (dd, J=8.4 and 2.0 Hz, 1H), 7.56 (d, J=2.0 Hz, 1H), 7.04 (d, J=8.5 Hz, 1H), 4.82 (d, J=2.4 Hz, 2H), 3.92 (s, 3H), 3.89 (s, 3H) and 2.53 (t, J=2.4 Hz, 1H); LCMS C₁₂H₁₂O₄Rt=6.044 min, ESI⁺ m/z=221.1 (M+H).

### 5-Methoxy-2-nitro-4-(prop-2-yn-1-yloxy)benzoic acid (12)

Under argon, at −5° C., to a suspension of (11) (440 mg, 2 mmol) in a mixture dichloromethane-acetic acid (3:1, 20 mL), was slowly added dropwise a solution of concentrated nitric acid (0.25 mL, 4 mmol) in dichloromethane (5 mL). The resulting mixture was allowed to warm to room temperature, stirred for 48 hours and concentrated under reduced pressure. The residue was recrystallized from methanol to afford 5-methoxy-2-nitro-4-(prop-2-yn-1-yloxy)benzoic acid (12) (408 mg, 77%) as fine yellow needles. ¹H NMR (250 MHz, CDCl₃) δ7.65 (s, 1H), 7.09 (s, 1H), 4.85 (d, J=2.6 Hz, 2H), 3.98 (s, 3H), 3.90 (s, 3H) and 2.60 (t, J=2.6 Hz, 1H); ¹³C NMR (63 MHz, CDCl₃) δ166.2, 153.1, 147.8, 140.6, 122.7, 111.1, 109.4, 77.5, 76.7, 57.1, 56.7 and 53.3; LCMS C₁₂H₁₁NO₆ Rt=6.126 min, ESI⁺ m/z=234.1 (M-OMe).

### 5-Methoxy-2-nitro-4-(prop-2-yn-1-yloxy)benzoic acid (13)

To a suspension of (12) (530 mg, 2 mmol) in tetrahydrofuran (10 mL), was added a 1M aqueous sodium hydroxide solution (10 mL, 20 mmol). The reaction mixture was heated at 50° C. for 2 hours and concentrated under reduced pressure to the water volume. The aqueous residue was successively diluted with cooled H2O (20 mL), washed with Et20 (2×10 mL), and acidified with concentrated aqueous HCl until pH1. The precipitate was collected by filtration, washed with water and dried at 60° C. overnight to afford 5-methoxy-2-nitro-4-(prop-2-yn-1-yloxy)benzoic acid (13) (430 mg, 86%) as a light yellow powder. ¹H NMR (250 MHz, MeOD) δ7.65 (s, 1H), 7.29 (s, 1H), 4.90 (d, J=2.4 Hz, 2H), 3.96 (s, 3H), 3.08 (t, J=2.4 Hz, 1H); ¹³C NMR (63 MHZ, MeOD) δ168.3, 153.8, 148.9, 141.8, 123.9, 112.2, 110.3, 78.1, 77.8, 57.8, 57.0. LCMS C₁₁H₉NO₆ Rt=5.234 min, ESI+ m/z=234.1 (M-OH), 274.0 (M+Na), ESI− m/z=250.1 (M-H).

### 1-(5-Methoxy-2-nitro-4-(prop-2-yn-1-yloxy)-benzoyl)-(2S)-4-methylenepyrrolidine-2-carboxylic acid methyl ester (14)

Under argon, to a suspension of (13) (377 mg, 1.5 mmol) in dichloromethane (3 mL), was successively added dimethylformamide (1 drop) and oxalyl chloride (0.4 mL, 4.5 mmol). The reaction mixture was stirred at room temperature for 12 hours and concentrated under reduced pressure. The corresponding acid chloride was suspended in dichloromethane (5 mL) and the suspension was added dropwise, at 0° C., to a mixture of (S)-4-methylenepyrrolidine-2-carboxylic acid methyl ester hydrochloride (34) (266 mg, 1.5 mmol) and triethylamine (0.46 mL, 3.3 mmol) in dichloromethane (5 mL). After addition, the resulting mixture was stirring at 0° C. for additional 15 minutes, then, successively diluted with CH2Cl2 (50 mL), washed with aqueous 1N HCl solution (3×20 mL), saturated aqueous Na2CO3 (20 mL) solution, and dried over Na2SO4. The solvent was distillated off under reduced to afford 1-(5-methoxy-2-nitro-4-(prop-2-yn-1-yloxy)-benzoyl)-(2S)-4-methylenepyrrolidine-2-carboxylic acid methyl ester (14) (530 mg, 94%) as a light brown powder. ¹H NMR (250 MHz, CDCl₃) rotamers; ¹³C NMR (63 MHz, CDCl₃) rotamers; LCMS C₁₈H₁₈N₂O₇ Rt=6.183 min, ESI+ m/z=375.0 (M+H).

### 1-(5-methoxy-2-nitro-4-(prop-2-yn-1-yloxy)-benzoyl)-(2S)-4-methylenepyrrolidine-2-formaldehyde (15)

Under argon, at −78° C., to a solution of (14) (530 mg, 1.42 mmol) in dichloromethane (15 mL), was added dropwise a solution of 1N diisobutylaluminium hydride in hexane (9.91 mL, 9.94 mmol). After addition, the reaction mixture was stirred at −78° C. for 15 min and quenched by addition of MeOH (10 mL). After the mixture was stirred for an additional 15 min, an aqueous solution of 1N HCl (15 mL) was added and the resulting solution was allowed to stir at 78° C. for an additional 15 min. The aqueous layer was extracted with CH₂Cl₂ (2×30 mL) and combined organic layers were washed with saturated aqueous Na₂CO₃ (2×20 mL) then dried over Na₂SO₄. The solvent was distillated off under reduced pressure and the residue was purified by column chromatography, eluent CH₂Cl₂—MeOH (97:3) to afford 1-(5-methoxy-2-nitro-4-(prop-2-yn-1-yloxy)-benzoyl)-(2S)-4-methylenepyrrolidine-2-formaldehyde (15) (470 mg, 89%) as a yellow powder. ¹H NMR (250 MHz CDCl₃) rotamers; ¹³C NMR (63 MHz, CDCl₁) rotamers; LCMS C₁₇H₁₆N₂O₆ Rt=5.488 min, ESI+ m/z=345.1 (M+H).

### (11aS)-7-Methoxy-8-(prop-2-yn-1-yloxy)-2-methylene-1,2,3,11a-tetrahydro-5H-pyrrolo[2,1-c][1,4]benzodiazepin-5-one (16)

At room temperature, to a solution of (15) (103 mg, 0.3 mmol) in a mixture tetrahydrofuran-water (v/v, 5:3, 0.03 M, 10 mL) was added sodium hydrosulfite (418 mg, 2.4 mmol) portionwise within 2 min. The resulting mixture was stirred for 1.5 hours, then quenched with MeOH (10 mL) and concentrated under reduced pressure. The solid residue was suspended in anhydrous methanol (10 mL) and, to the resulting solution, was added dropwise AcCl (0.21 mL, 3 mmol). After stirring for 20 min, the solution was filtrated and the precipitate washed with MeOH (5 mL). The yellow filtrate was stirred for additional 1.5 hours and then quenched with saturated aqueous solution of Na₂CO₃ (10-20 mL) until pH basic. The mixture was concentrated under reduced pressure until water volume, diluted with H₂O (50 mL) and extracted with EtOAc (3×20 mL). The organic layers were combined and dried over Na₂SO₄. The solvent was distillated off under reduced pressure and the residue was purified by column chromatography, gradient hexane-EtOAc (1:3 to 1:5) to afford (11aS)-7-methoxy-8-(prop-2-yn-1-yloxy)-2-methylene-1,2,3,11a-tetrahydro-5H-pyrrolo[2,1-c][1,4]benzodiazepin-5-one (16) (70 mg, 79%) as a light orange solid. ¹H NMR (250 MHz, CDCl₃) δ (imine selected data) ¹H NMR (250 MHz, CDCl₃) δ7.68 (d, J=4.5 Hz, 1H), 7.51 (s, 1H), 6.96 (s, 1H), 5.20-5.15 (m, 2H), 4.81 (d, J=2.4 Hz, 2H), 4.28-4.26 (m, 2H), 3.94 (s, 3H), 3.92-3.87 (m, 1H), 3.20-3.01 (m, 1H), 3.00-2.83 (m, 1H) and 2.53 (t, J=2.4 Hz, 1H); LCMS C₁₇H₁₆N₂O₃ Rt=5.117 min, ESI+ m/z=297.1 (M+H), 315.1 (M+H₂O).

### Example 5. Preparation of Precursor (8)

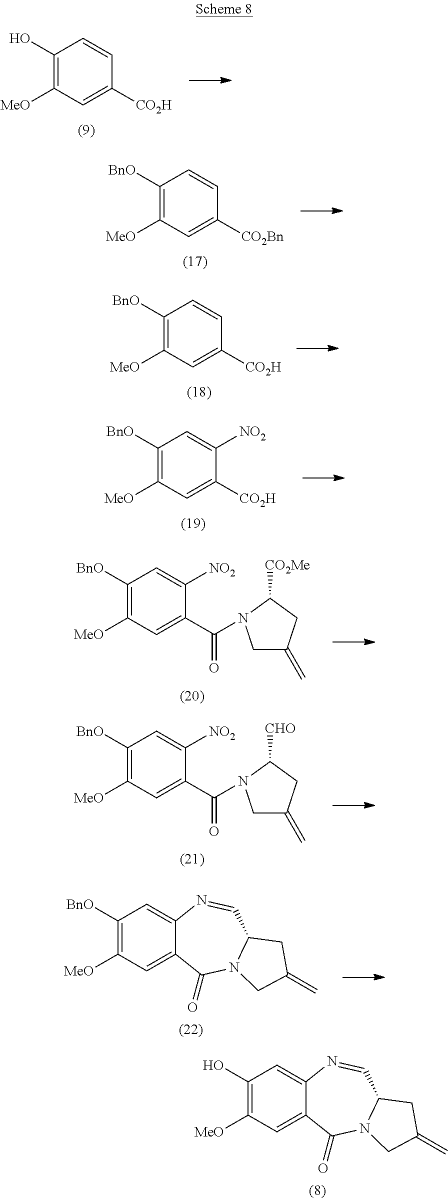

### 4-Benzyloxy-3-methoxybenzoic acid benzyl ester (17)

Under argon, at room temperature, to a suspension of isovanillic acid (9) (20.0 g, 119 mmol) and potassium carbonate (49.3 g, 357 mmol) in dimethylformamide (400 mL) was added benzyl bromide (35 mL, 286 mmol). The reaction mixture was stirred overnight at room temperature, and then poured into water (350 mL) and extracted with EtOAc (3×100 mL). The organic layers were combined, dried over Na₂SO₄, and concentrated under reduced pressure. The yellow-brown oil residue was purified by crystallization from cyclohexane, under vigorous stirring, to afford 4-benzyloxy-3-methoxybenzoic acid benzyl ester (17) (41.0 g, quantitative) as colorless powder. ¹H NMR (250 MHz, CDCl₃) δ7.65 (dd, J=8.4 and 2.0 Hz, 1H), 7.60 (d, J=2.0 Hz, 1H), 7.47-7.31 (m, 10H), 6.88 (d, J=8.4 Hz, 1H), 5.34 (s, 2H), 5.22 (s, 2H), 3.93 (s, 3H); LCMS C₂₂H₂₀O₄ Rt=7.730 min, ESI+ m/z=349.0 (M+H).

### 4-Benzyloxy-3-methoxybenzoic acid (18)

To a suspension of (17) (41.3 g, 119 mmol) in a mixture water-ethanol (1:1, 200 mL), was added sodium hydroxide (9.4 g, 238 mmol). The reaction mixture was refluxed for 2 hours and concentrated under reduced pressure to the water volume. The aqueous residue was successively diluted in cooled H₂O (100 mL), washed with Et₂O (2×20 mL), acidified with concentrated aqueous HCl and then extracted with EtOAc (3×30 mL). The organic layers were combined, washed with water (20 mL) and dried over Na₂SO₄. The solvent was removed under reduced pressure to afford 4-benzyloxy-3-methoxybenzoic acid (18) (29.4 g, 96%). ¹H NMR (250 MHz, CDCl₃) δ7.70 (dd, J=8.4 and 1.9 Hz, 1H), 7.61 (d, J=1.9 Hz, 1H), 7.46-7.32 (m, 5H), 6.93 (d, J=8.4 Hz, 1H), 5.24 (s, 2H) and 3.95 (s, 3H); LCMS C₁₅H₁₄O₄ Rt=6.252 min, ESI+ m/z=258.9 (M+H).

### 4-Benzyloxy-5-methoxy-2-nitrobenzoic acid (19)

Under argon, to a solution of (18) (30 g, 116 mmol) in a mixture dichloromethane-acetic acid (4:1, 350 mL), was added dropwise concentrated nitric acid (15 mL, 232 mmol). The reaction mixture was stirred at room temperature for 72 hours and concentrated under reduced pressure to acetic acid volume. The residue was poured into EtOH (200 mL), the precipitate was collected by filtration and recrystallized from EtOAc-hexane to afford 4-benzyloxy-5-methoxy-2-nitrobenzoic acid (19) (21.8 g, 62%). ¹H NMR (250 MHZ, CDCl₃) δ7.46 (s, 1H), 7.44-7.30 (m, 5H), 7.21 (s, 1H), 5.23 (s, 2H) and 3.99 (s, 3H); LCMS C₁₅H₁₃NO₆ Rt=6.299 min, ESI+ m/z=285.9 (M-OH), 303.9 (M+H), 325.8 (M+Na).

### 1-(4-Benzyloxy-5-methoxy-2-nitrobenzoyl)(2S)-4-methylenepyrrolidine-2-carboxylic acid methyl ester (20)

Under argon, to a suspension of the (19) (2.6 g, 8.6 mmol) in dichloromethane (10 mL), was successively added dimethylformamide (1 drop) and oxalyl chloride (2 mL, 25.8 mmol). The reaction mixture was stirred at room temperature for 2 hours and concentrated under reduced pressure. The corresponding acid chloride was suspended in dichloromethane (10 mL) and the suspension was added dropwise, at 0° C., to a mixture of (S)-4-methylenepyrrolidine-2-carboxylic acid methyl ester hydrochloride (1.39 g, 7.74 mmol) and triethylamine (6 mL) in dichloromethane (20 mL). After addition, the resulting mixture was stirring at 0° C. for additional 15 minutes, then at room temperature for 8 hours. The solvent was distillated off under reduced pressure and the residue successively suspended in H₂O (150 mL) and extracted with EtOAc (3×60 mL). The combined organic layers were washed with brine (2×40 mL), and dried over Na₂SO₄. After distillation under reduced pressure, the oil residue was purified by column chromatography, eluent petroleum cyclohexane-EtOAc (1:1) to afford 1-(4-benzyloxy-5-methoxy-2-nitrobenzoyl)(2S)-4-methylenepyrrolidine-2-carboxylic acid methyl ester (20) (2.95 g, 81%) as a yellow oil which crystallize at room temperature. ¹H NMR (250 MHz, CDCl₃) rotamers; LCMS C₂₂H₂₂N₂O₇ Rt=6.757 min, ESI+ m/z=426.9 (M+H).

### 1-(4-Benzyloxy-5-methoxy-2-nitrobenzoyl)-(2S)-4-methylenepyrrolidine-2-formaldehyde (21)

Under argon, at −78° C., to a solution of (20) (2.21 g, 5.2 mmol) in dichloromethane (60 mL), was added dropwise a solution of 1N diisobutylaluminium hydride in hexane (15.6 mL, 15.6 mmol). After addition, the reaction mixture was stirred at −78° C. for 1.5 hours and quenched by addition of MeOH (2 mL). After the mixture was stirred for an additional 15 min, an aqueous solution of 1N HCl (15 mL) was added and the resulting solution was allowed to stir at 0° C. for an additional 15 min. The aqueous layer was extracted with CH₂Cl₂ (3×50 mL) and combined organic layers were washed with brine (40 mL) then dried over MgSO₄. The solvent was distillated off under reduced pressure to afford 1-(4-benzyloxy-5-methoxy-2-nitrobenzoyl)-(2S)-4-methylenepyrrolidine-2-formaldehyde (21) (1.74 g, 84%) as a yellow solid. ¹H NMR (250 MHz, CDCl₃) δ7.65 (d, J=4.4 Hz, 1H), 7.52 (s, 1H), 7.47-7.30 (m, 5H), 6.84 (s, 1H), 5.25-5.14 (m, 4H) , 4.28 -4.26 (m, 2H), 3.96 (s, 3H), 3.76-3.61 (m, 1H), 3.11 (dd, J=15.5 and 9.0 Hz, 1H) and 2.92 (d, J=15.5 Hz, 1H);. LCMS C₂₁H₂₀N₂O₃ Rt=6.001 min, ESI+ m/z=349.0 (M+H), 367.0 (M+H).

### (11aS)-1,2,3,11a-Tetrahydro-7-methoxy-2-methylene-8-(phenylmethoxy)-5H-pyrrolo[2,1-c][1,4]benzodiazepin-5-one (22)

At room temperature, to a solution of (21) (1.74 g, 4.8 mmol) in a mixture tetrahydrofuran-water (v/v, 5:3, 0.06 M, 80 mL) was added sodium hydrosulfite (6.5 g, 38.4 mmol) portion wise. The resulting mixture was stirred overnight and subsequently concentrated under reduced pressure. The residue was suspended in anhydrous methanol (60 mL) and acidified with aqueous solution 6N HCl until pH=2. After stirring for 1 hour, the solution was concentrated under reduced pressure until few volume and the residue diluted with EtOAc (100 mL). The organic layer was successively washed with saturated aqueous NaHCO₃ (3×40 mL), brine (20 mL), and then dried over Na₂SO₄. The solvent was distillated off under reduced pressure to give (11aS)-1,2,3,11a-tetrahydro-7-methoxy-2-methylene-8-(phenylmethoxy)-5H-pyrrolo[2,1-c][1,4]benzodiazepin-5-one (22) (1.37 g, 88%) as a hygroscopic light orange solid. ¹H NMR (250 MHz, CDCl₃) δ7.65 (d, J=4.4 Hz, 1H), 7.52 (s, 1H), 7.47-7.30 (m, 5H), 6.84 (s, 1H), 5.25-5.14 (m, 2H), 5.17 (s, 2H), 4.28 -4.26 (m, 2H), 3.96 (s, 3H), 3.76-3.61 (m, 1H), 3.11 (dd, J=15.5 and 9.0 Hz, 1H) and 2.92 (d, J=15.5 Hz, 1H); ¹³C NMR (63 MHz, CDCl₃). LCMS (SB015P3) C21H20N2O3 Rt=6.001 min, ESI+ m/z=349.0 (M+H), 367.0 (M+H).

### (11aS)-8-Hydroxy-7-methoxy-2-methylene-1,2,3,11a-tetrahydro-5H-pyrrolo[2,1-c][1,4]benzodiazepin-5-one (8)

At 0° C., to a solution of (22) (1.37 g, 3.93 mmol) in dichloromethane (70 mL) was added methanesulfonic acid (10 mL). The mixture was stirred at 0° C. for 10 min, then allowed to warm to room temperature and stirred for an additional 2 hours at r.t. The resulting mixture was diluted with CH₂Cl₂ then basified with cold 1N aqueous NaHCO₃ solution until pH4, and filtrated. The aqueous layer was extracted with CH₂Cl₂ (3×60 mL) and the combined organic layers were dried over Na₂SO₄. The solvent was distillated off under reduced pressure to give the (11aS)-8-hydroxy-7-methoxy-2-methylene-1,2,3,11a-tetrahydro-5H-pyrrolo[2,1-c][1,4]benzodiazepin-5-one (8) (980 mg, 97%) as a yellow solid. ¹H NMR (400 Hz, CDCl₃): δ7.69 (d, J=4.2 Hz, 1H), 7.50 (s, 1H), 6.89 (s, 1H), 5.19 (broad s, 1H), 5.16 (broad s, 1H), 4.28 (s, 2H), 3.97 (s, 3H), 3.91-3.87 (m, 1H), 3.13-2.96 (m, 2H); LCMS C₁₄H₁₄N₂O₃ Rt=4.461 min, ESI⁺ m/z=258.9 (M+H), 277.0 (M+H₂O).

### Example 6. Preparation of Precursor (1)

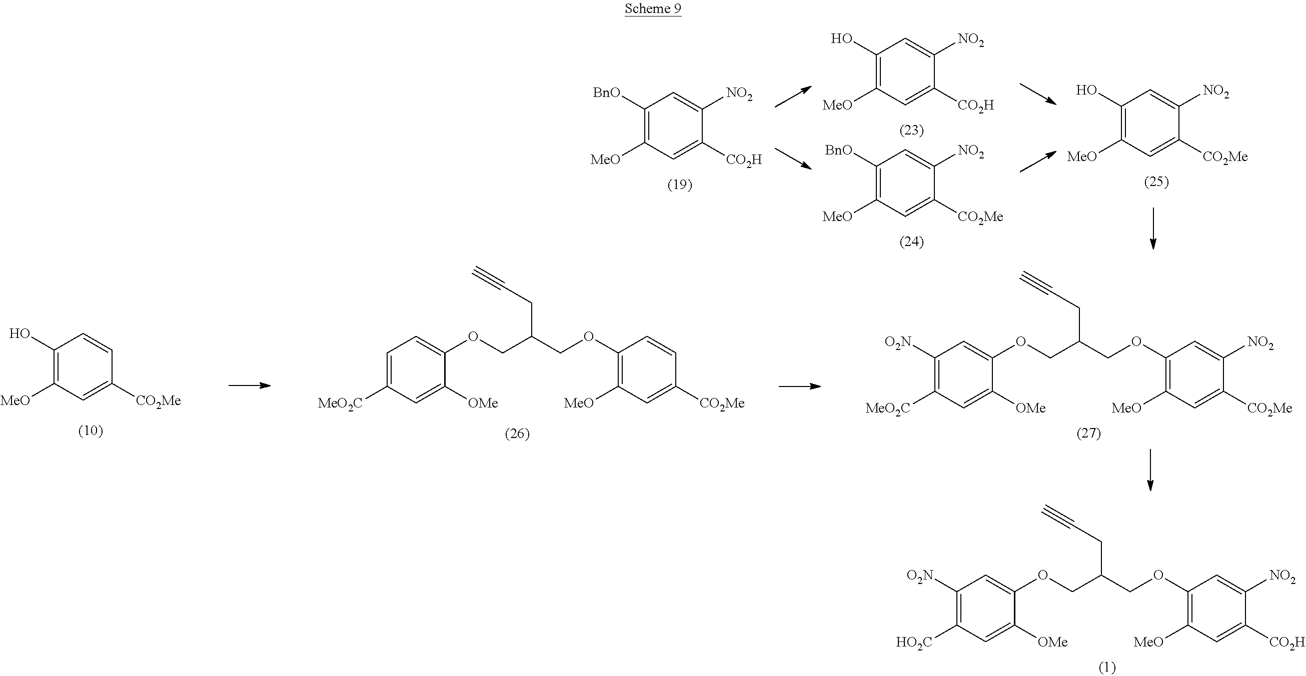

### 4-Hydroxy-5-methoxy-2-nitrobenzoic acid (23)

Under argon, at room temperature, to a suspension of (19) (6.6 g, 22 mmol) in dichloromethane (70 mL), was injected methanesulfonic acid (10 mL, 176 mmol). The reaction mixture was vigorously stirred for 1 hour, then successively cooled to 0° C. and basified with 10% aqueous NaOH solution until pH7. The precipitate was collected by filtration to afford 4-hydroxy-5-methoxy-2-nitrobenzoic acid (23) (1.0 g, 22%). LCMS C₈H₇NO₆ Rt=2.736 min, ESI+ m/z=196.9 (M-H2O), 213.8 (M+H), 235.9 (M+Na).

### 4-Benzyloxy-3-methoxy-2-nitrobenzoic acid methyl ester (24)

Under argon, at room temperature, to a suspension of (19) (4.0 g, 131 mmol) in absolute methanol (100 mL) was added dropwise thionyl chloride (1.9 mL, 262 mmol). The resulting mixture was stirred at 60° C. for 96 hrs then concentrated under reduced pressure until few volume. The residue was allowed to crystallize at −20° C. and the solid was collected by filtration affording 4-benzyloxy-3-methoxy-2-nitrobenzoic acid methyl ester (24) (3.36 g, 80%) as a white crystal. LCMS C₁₆H₁₅NO₆ Rt=6.862 min, ESI+ m/z=318.9 (M+H), 285.9 (M-OMe).

### 4-Hydroxy-5-methoxy-2-nitrobenzoic acid methyl ester (25)

Method A. Under argon, at room temperature, to a suspension of (23) (980 mg, 4.7 mmol) in absolute methanol (70 mL) was added dropwise thionyl chloride (0.5 mL, 7.1 mmol). The resulting mixture was stirred at 60° C. for 6 days then concentrated under reduced pressure. The residue was diluted with CH2Cl2 (100 mL) washed with 1N aqueous HCl solution (2×20 mL) and dried over Na2SO4. The solvent was distillated off under reduced pressure affording 4-hydroxy-3-methoxybenzoic acid methyl ester (25) (870 mg, 83%) as a light yellow solid. LCMS C₉H₉NO₆ Rt=5.610 min, ESI+ m/z =195.9 (M-OMe), 228.0 (M+H), 249.9 (M+Na).

Method B. Under argon, at 0° C., to a suspension of (24) (2.86 g, 90 mmol) in dichloromethane (20 mL), was slowly added dropwise a solution of methanesulfonic acid (1.22 mL, 189 mmol) in dichloromethane (5 mL). After addition, the resulting mixture was vigorously stirred at room temperature for 2.5 hours and concentrated under reduced pressure until few volume. The viscous residue was purified by flash chromatography, eluent CH2Cl2 to afford 4-hydroxy-5-methoxy-2-nitrobenzoic acid methyl ester (25) (1.88 g, 92%) as a yellow solid.

### 4,4′-((2-(Prop-2-yn-1-yl)propane-1,3-diyl)bis(oxy))bis(3-methoxybenzoic acid methyl ester) (26)

Method A. Under argon, at 0° C. to a solution of (10) (900 mg, 4.9 mmol) and triphenylphosphine (3.24 g, 12.4 mmol) in anhydrous tetrahydrofuran (10 mL) was added dropwise diethyl azodicarboxylate (1.95 mL, 9.9 mmol) over 5 min. After addition, the resulting mixture was vigorously stirred at 0° C. for 30 min and a solution of 2-(prop-2-ynyl)propane-1,3-diol (170 mg, 2.4 mmol) in anhydrous tetrahydrofuran (3 mL) was added dropwise. The reaction mixture was stirred at 0° C. for an additional 1 hr and concentrated under reduced pressure. The crude residue was purified by column chromatography, eluent petroleum ether-EtOAc (3:1) to afford the 4,4′-((2-(Prop-2-yn-1-yl)propane-1,3-diyl)bis(oxy))bis(3-methoxybenzoic acid methyl ester) (26) (1 g, quantitative) as a white crystal. ¹H NMR (400 MHz, CDCl₃) δ7.63 (d, J=8.2 Hz, 2H), 7.53 (s, 2H), 6.95 (d, J=8.2 Hz, 2H), 4.26 (m, 4H), 3.88 (s, 6H), 3.87 (s, 6H), 2.69 (sept, J=5.6 Hz, 1H), 2.62 (dd, J=5.6 and 2.7 Hz, 2H) and 2.02 (t, J=2.7 Hz, 1H); ¹³C NMR (100 MHz, CDCl₃) δ166.9, 152.3, 149.1, 123.5, 123.0, 112.5, 112.1, 81.3, 70.6, 68.1, 56.1, 52.1, 37.9 and 18.1. LCMS C₂₄H₂₆O₈ Rt=7.131 min, ESI+ m/z=411.0 (M-OMe).

Method B. Under argon, to a solution of (10) (149 mg, 0.81 mmol) in dry dimethylformamide (6 mL) was added 2-(prop-2-yn-1-yl)propane-1,3-diyl dimethanesulfonate (38) (106 mg, 0.39 mmol) and potassium carbonate (445 mg, 3.21 mmol). The resulting mixture was heated to 90° C. for 3 hours. The reaction mixture was allowed to cool at room temperature and then concentrated under reduced pressure. The residue was successively poured into water (5 mL) and extracted with CH₂Cl₂ (2×5 mL). The combined organic layers were dried over Na₂SO₄, and the solvent was distillated under reduced pressure. The residue was purified by column chromatography, eluent petroleum ether-EtOAc (7:3) to afford the 4,4′-((2-(Prop-2-yn-1-yl)propane-1,3-diyl)bis(oxy))bis(3-methoxybenzoic acid methyl ester) (26) (140 mg, 80%) as a white crystal.

### 4,4′-((2-(Prop-2-yn-1-yl)propane-1,3-diyl)bis(oxy))bis(5-methoxy-2-nitrobenzoic acid methyl ester) (27)

Method A. Under argon, at 0° C., to a solution of (25) (509 mg, 2.24 mmol) and triphenylphosphine (1.47 g, 5.60 mmol) in anhydrous tetrahydrofuran (10 mL) was added dropwise diethyl azodicarboxylate (0.88 mL, 4.48 mmol) over 5 min. After addition, the resulting mixture was vigorously stirred at 0° C. for 30 min and a solution of 2-(prop-2-ynyl)propane-1,3-diol (123 mg, 1.08 mmol) in anhydrous tetrahydrofuran (5 mL) was added dropwise. Stirring was maintained at 0° C. for an additional 1 hour, then the reaction mixture was concentrated under reduced pressure and the crude residue was purified by flash chromatography, gradient petroleum ether-EtOAc (7:3 to 1:1) to afford 4,4′-((2-(Prop-2-yn-1-yl)propane-1,3-diyl)bis(oxy))bis(5-methoxy-2-nitrobenzoic acid methyl ester) (27) (177 mg, 69%) as a light yellow powder. ¹H NMR (250 MHz, CDCl₃) δ7.52 (s, 2H), 7.06 (s, 2H), 4.30 (d, J=5.8 Hz, 4H), 3.94 (s, 6H), 3.91 (s, 6H), 2.71 (sept, J=5.8 Hz, 1H), 2.61 (dd, J=5.8 and 2.6 Hz, 2H) and 2.10 (t, J=2.6 Hz, 1H); ¹³C NMR (63 MHz, CDCl₃) δ166.1, 153.0, 149.5, 141.0, 122.0, 111.1, 108.6, 80.6, 71.0, 68.3, 56.5, 53.1, 37.9 and 17.8. LCMS C₂₄H₂₄N₂O₁₂ Rt=7.095 min, ESI+ m/z=500.9 (M-OMe).

Method B. Under argon, to a solution of (25) (3.2 g, 14.1 mmol) in dry dimethylformamide (180 mL) was added 2-(prop-2-yn-1-yl)propane-1,3-diyl dimethanesulfonate (1.84 g, 6.8 mmol) and potassium carbonate (7.89 g, 57.0 mmol). The resulting mixture was heated to 75° C. for 6 hours. The reaction mixture was allowed to cool to room temperature and then concentrated under reduced pressure. The residue was successively poured into water (100 mL) and extracted with CH₂Cl₂ (2×100 mL). The combined organic layers were dried over Na₂SO₄, and the solvent was distillated under reduced pressure. The residue was purified by flash chromatography, eluent cyclohexane-EtOAc (7:3) to afford 4,4′-((2-(prop-2-yn-1-yl)propane-1,3-diyl)bis(oxy))bis(5-methoxy-2-nitrobenzoic acid methyl ester) (27) (2.89 g, 80%) as a light yellow powder.

Method C. Under argon, to a suspension of (26) (100 mg, 0.23 mmol) in a mixture dichloromethane-acetic acid (4:1, 5 mL), was added dropwise concentrated nitric acid (0.375 mL, 9.20 mmol). The reaction mixture was stirred at room temperature for 120 hours and concentrated under reduced pressure. The residue was purified by flash chromatography, eluent cyclohexane-EtOAc (4:2) to afford 4,4′-((2-(Prop-2-yn-1-yl)propane-1,3-diyl)bis(oxy))bis(5-methoxy-2-nitrobenzoic acid methyl ester) (27) (116 mg, 97%) as yellow powder.

### 4,4′-((2-(Prop-2-yn-1-yl)propane-1,3-diyl)bis(oxy))bis(5-methoxy-2-nitrobenzoic acid) (1)

To a suspension of (27) (816 mg, 1.53 mmol) in ethanol (15 mL), was added a 1M aqueous sodium hydroxide solution (15 mL, 15.3 mmol). The reaction mixture was heated at 50° C. for 2 hours and concentrated under reduced pressure to the water volume. The aqueous residue was successively diluted with cooled H2O (50 mL), washed with CH2C2 (3×20 mL), and acidified with concentrated aqueous HCl until pH1. The precipitate was collected by filtration, washed with water and dried at 80° C. overnight to afford 4,4′-((2-(prop-2-yn-1-yl)propane-1,3-diyl)bis(oxy))bis(5-methoxy-2-nitrobenzoic acid) (1) (613 mg, 79%) as a yellow powder. ¹H NMR (250 MHz, DMSO-D₆) δ7.62 (s, 2H), 7.27 (s, 2H), 4.25 (m, 4H), 3.89 (s, 6H), 2.93 (m, 1H); LCMS C₂₂H₂₀N₂O₁₂ Rt=6.069 min, ESI+ m/z=486.9 (M-OH), ESI− m/z=503.8 (M-H).

### Example 7. Preparation of Compounds (29-34)

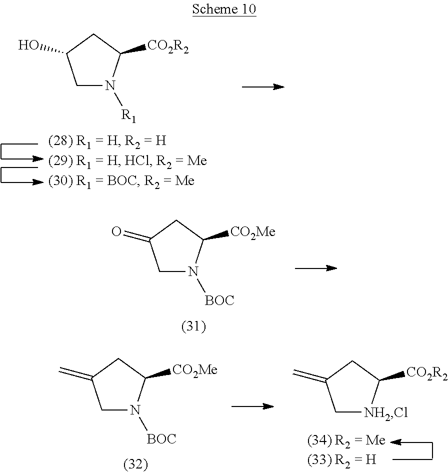

### (2S, 4R)-Methyl 4-hydroxypyrrolidine-2-carboxylate hydrochloride (29)

At 0° C., to a solution of trans-4-hydroxy-L-proline (28) (20.0 g, 0.15 mol) in methanol (150 mL) was added dropwise thionyl chloride (16.6 mL, 0.23 mol). After addition, the resulting mixture was allowed to warm to room temperature and stirred overnight. The reaction mixture was concentrated under reduced pressure and the residue was crystallized from Et₂O (100 mL) to afford the (2S, 4R)-methyl 4-hydroxypyrrolidine-2-carboxylate hydrochloride (29) (27.5 g, quantitative) as white crystals. ¹H NMR (250 MHz, MeOD) δ4.64-4.57 (m, 2H), 3.86 (s, 3H), 3.43 (dd, J=12.3 and 3.6 Hz, 1H), 3.33-3.27 (m, 1H), 2.42 (ddt, J=13.7, 7.6, and 1.6 Hz, 1H) and 2.19 (ddd, J=13.7, 10.7, and 4.1 Hz, 1H); LCMS (SB001P1) C₆H₁₁NO₃ Rt=0.967 min, ESI+ m/z=145.9 (M+H).

### (2S, 4R)-1-tert-Butyl 2-methyl 4-hydroxypyrrolidine-1,2-dicarboxylate (30)

Under argon, at 0° C., to a suspension of (29) (27.6 g, 0.15 mol) in acetonitrile (200 mL) was successively added triethylamine (63.6 mL, 0.47 mol) and di-tert-butyl dicarbonate (33.2 g, 0.15 mol). The resulting mixture was stirred at room temperature overnight. After filtration, the filtrate was concentrated under reduced pressure and the residue poured into CH₂Cl₂ (300 mL). The organic layer was washed with 1N aqueous NaHSO₄ solution (4×100 mL), dried over Na₂SO₄, and concentrated under reduced pressure to give the (2S, 4R)-methyl 1-(tert-butyloxycarbonyl)-4-hydroxypyrrolidine-2-carboxylate (30) (36.7 g, 99%) as light yellow oil. ¹H NMR (250 MHz, CDCl₃, rotamers) δ4.49-4.36 (m, 2H), 3.72 (s, 3H), 3.60-3.43 (m, 2H), 2.24-2.17 (m, 1H), 2.11-2.01 (m, 2H), 1.45 and 1.40 (2×s, 9H); LCMS C₁₁H₁₉NO₅ Rt=5.101 min, ESI+ m/z=146.1 (M+2H-Boc).

### (S)-Methyl 1-tert-butyloxycarbonyl)-4-oxopyrrolidine-2-carboxylate (31)

At 0° C., to a solution of (30) (30 g, 0.12 mol) in dichloromethane (300 mL) were successively added trichloroisocyanuric acid (28.1 g, 0.12 mol) and 2,2,6,6-tetramethyl-1-piperidinyloxy free radical (180 mg, 1.15 mmol). The resulting mixture was allowed to warm to room temperature for approximatively 15 min, until starting material was completely consumed, monitored by TLC, eluent cyclohexane-EtOAc (7:3). After filtration, the organic layer was successively washed with saturated aqueous Na₂CO₃ solution (200 mL), 1N aqueous HCl solution, brine, then dried over Na₂SO₄. The solvent was distillated under reduced pressure affording the (S)-methyl 1-(tert-butyloxycarbonyl)-4-oxopyrrolidine-2-carboxylate (31) (28.1 g, 94%) as orange oil. ¹H NMR (250 MHz, CDCl₃, rotamers) δ4.74 (m, 1H), 3.87 (m, 2H), 3.74 (s, 3H), 3.07-2.78 (m, 1H), 2.56 (m, 1H), 1.45 and 1.43 (2×s, 9H); LCMS C₁₁H₁₇NO₅ Rt=5.702 min, ESI+ m/z=144.0 (M+2H-Boc).

### (S)-Methyl 1-(-tert-butyloxycarbonyl)-4-methylenepyrrolidine-2-carboxylate (32)

At 0° C. to a suspension of methyltriphenylphosphonium bromide (35.26 g, 98.7 mmol) in freshly distillated THF (600 mL) was added potassium-tert-butoxide (11.1 g, 98.7mmol) portionwise. After stirring at 0° C. for 30 min, the solution was allowed to warm to room temperature for extra 30 min. then cooled down to 0° C. At this temperature, a solution of (31) (16.0 g, 65.8 mmol) in freshly distillated THF (200 mL) was added drop by drop, over 1 hour. After addition, the reaction mixture was stirred at room temperature for 4 hours and then concentrated under reduced pressure. The filtrate was triturated with a mixture of petroleum ether-Et₂O (8:2, 200 mL) and filtrated through a short pad of celite. The filtrate was concentrated under reduced pressure and the residue was purified by column chromatography, eluent cyclohexane-EtOAc (8:2) to afford the (S)-methyl 1-(tert-butyloxycarbonyl)-4-methylenepyrrolidine-2-carboxylate (32) (10.9 g, 68%) as yellow oil. ¹H NMR (250 MHz, CDCl₃, rotamers) δ5.01-4.98 (m, 2H), 4.52-4.36 (m, 1H), 4.12-4.04 (m, 2H), 3.71 (s, 3H), 3.02-2.88 (m, 1H), 2.64-2.57 (m, 1H), 1.46 and 1.41 (2×s, 9H); LCMS C₁₂H₁₉NO₄ Rt=6.490 min, ESI+ m/z=142.9 (M+2H-Boc).

### (S)-4-Methylenepyrrolidine-2-carboxylic acid hydrochloride (33)

At 0° C., to a suspension of (32) (6.5 g, 27.0 mmol) in ethyl acetate (40 mL) was added an aqueous 10N hydrochloric acid solution (10 mL). The mixture was stirred for 2 hours and concentrated under reduced pressure, at 60° C., until dryness. The solid residue was purified by recrystallization from a mixture MeOH-Et₂O to afford the (S)-4-methylenepyrrolidine-2-carboxylic acid hydrochloride (33) (3.47 g, 79%) as a light brown powder. ¹H NMR (250 MHz, DMSO-D₆) δ5.16-5.14 (m, 2H), 4.45 (t, J=8.3 Hz, 1H), 3.88-3.84 (m, 2H), 2.95 (dd, J=16.1 and 8.3 Hz, 1H) and 2.71 (dd, J=16.1 and 8.3 Hz, 1H); LCMS C₆H₉NO₂ Rt=0.896 min, ESI+ m/z=128.2 (M+H).

### (S)-Methyl 4-methylenepyrrolidine-2-carboxylate hydrochloride (34)

Method A. At 0° C., to a suspension of (32) (6.8 g, 28.2 mmol) in ethyl acetate (40 mL) was added an aqueous 10N hydrochloric acid solution (10 mL). The mixture was stirred for approximatively 1 hour, until complete consumption of starting material, and concentrated at room temperature under vacuum. The solid residue was purified by recrystallization from MeOH to afford the (S)-methyl 4-methylenepyrrolidine-2-carboxylate hydrochloride (34) (3.3 g, 66%) as a light brown powder [α]²⁰_{D}−15.02 (c=1, H₂O). ¹H NMR (250 MHz, DMSO-D₆) δ5.15-5.09 (m, 2H), 4.59 (t, J=8.4 Hz, 1H), 3.89-3.82 (m, 2H), 3.76 (s, 3H), 2.96 (dd, J=16.4 and 8.4 Hz, 1H) and 2.72 (dd, J=16.4 and 8.4 Hz, 1H); LCMS C₇H₁₁NO₂ Rt=1.600 min, ESI+ m/z=142.1 (M+H).

Method B. To a solution of (33) (1.63 g, 10 mmol) in methanol (50 mL) was added drop by drop thionyl chloride (0.73 mL, 20 mmol). After addition, the resulting mixture was refluxed overnight and then concentrated under reduced pressure until few volume. The residue was crystallized from Et20 (50 mL) and the solid collected by filtration to afford the (S)-methyl 4-methylenepyrrolidine-2-carboxylate hydrochloride (34) (1.57 g, 88%) as white needles.

### Example 8. Preparation of Compounds (36-38)

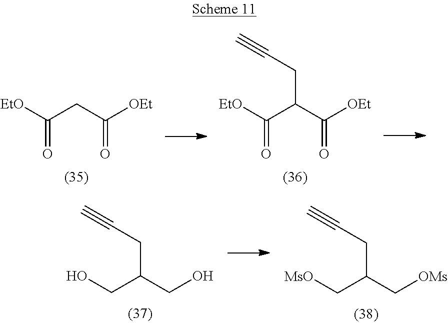

### Diethyl 2-(prop-2-yn-1-yl)malonate (36)

Under argon, at 50° C., to diethyl malonate (12.8 g, 79.9 mmol) was injected a solution, freshly prepared, of sodium ethanolate (1.66 g of sodium, 30 mL absolute ethanol). The reaction mixture was stirred for 1.5 hours at 50° C. and then allowed to cold to room temperature before adding a 80% solution of propargyl bromide in toluene (8.6 mL, 79.9 mmol). The resulting mixture was stirred at room temperature for 3 hours and concentrated under reduced pressure. The residue was poured into H2O (50 mL) and extracted with Et2O (3×30 mL). The organic layers were combined, dried over MgSO₄, and the solvent was distillated under reduced pressure. The crude product was purified under reduced pressure by fractional distillation, to afford the diethyl 2-(prop-2-yn-1-yl)malonate (36) (6.63 g, 47%) as colorless oil (bp 82° C./10 mmHg). ¹H NMR (250 MHz, CDCl₃) 8 4.22 (q, J=7.1 Hz, 4H), 3.55 (t, J=7.7 Hz, 1H), 2.77 (dd, J=7.7, 2.1 Hz, 2H), 2.00 (t, J=2.6 Hz, 1H), 1.27 (t, J=7.1 Hz, 6H).

### 2-(Prop-2-yn-1-y)propane-1.3-diol (37)

Under argon, at 0° C., to a suspension of lithium aluminum hydride (2.96 g, 80 mmol) in dry diethyl ether (150 mL), was added dropwise a solution of (36) (7.75 g, 39 mmol) in dry diethyl ether (10 mL) over 15 min. The reaction mixture was allowed to warm to room temperature and stirred overnight. The reaction was quenched with a saturated Na₂SO₄ aqueous solution (100 mL), and extracted with Et₂O (40 mL). The combined organic layers were washed with brine (20 mL), dried over Na₂SO₄, and the solvent was distillated under reduced pressure. The residue was purified by column chromatography, eluent cyclohexane-EtOAc (gradient 1:1 then 0:1) to afford 2-(prop-2-yn-1-yl)propane-1,3-diol (37) (1.81 g, 40%) as colorless oil. ¹H NMR (250 MHz, CDCl₃) δ3.82 (dd, J=10.9 and 4.7 Hz, 2H), 3.76 (dd, J=10.9 and 6.2 Hz, 2H), 2.29 (dd, J=6.2 and 2.6 Hz, 2H), 2.00 (t, J=2.6 Hz, 1H) and 1.95 (sept, J=6.2 Hz, 1H); ¹³C NMR (63 MHZ, CDCl₃) δ82.2, 70.0, 64.0, 41.4 and 17.5. ¹H NMR

### 2-(prop-2-yn-1-yl)propane-1,3-diyl dimethanesulfonate (38)

Under argon, at 0° C., to a solution of (36) (800 mg, 7.0 mmol) in dichloromethane (40 mL) were successively added dropwise methanesulfonyl chloride (1.2 mL, 15.4 mmol) and triethylamine (3.2 mL, 22.4 mmol). After addition, the mixture was stirred at 0° C. for 30 min, then at room temperature for 3 hours. The reaction mixture was poured into H2O (40 mL), and the organic layer was washed with H2O (40 mL), dried over Na2SO4, and concentrated under reduced pressure to afford 2-(prop-2-yn-1-yl)propane-1,3-diyl dimethanesulfonate (38) (1.79 g, 95%) as light brown oil. ¹H NMR (250 MHz, MeOD) δ4.46-4.24 (m, 4H), 3.12 (s, 6H) and 2.52-2.32 (m, 4H); ¹³C NMR (63 MHz, CDCl₃) δ79.3, 71.7, 67.5, 37.5, 37.4 and 17.3.

### Example 9: In Situ Detection of SJG-136-Click in Cells by Microscopy

### In Vivo Crosslinking Assay

HeLa cells were seeded onto 6 coverslips in 24-well cluster plates at 35000 cells/well. The next day, the following treatment were applied:
1. One well was treated with 2ml of 10 μM of EDU (252 g·mol⁻¹) in DMEM for 30 min at 37° C. as positive control.
2. One well was treated with 2ml of 10 μM of SJG-136 in DMEM for 15 min to 1 h at 37° C. as negative control.
3. The other wells were treated with 10 μM of SJG-136-click in 10 mL of DMEM for 15 min to 1 h at 37° C.

After treatments, cells were washed with PBS at 37° C., optionally preextracted with ice-cold CSK buffer (100 mM NaCl, 10 mM HEPES (pH=7.8), 3 mM MgCl₂, 300 mM sucrose, 0.5% Triton X-100) and 100 μg·mL⁻¹ RNase for 2 min on ice to remove non-chromatin bound protein, and fixed with 4% PAF/PBS for 12 min at room temperature. Cells are then optionally washed with PBS/0.1% Triton X-100 containing 100 μg·mL⁻¹ RNase for 30 min at room temperature. Finally, cells were blocked with 3% FBS in PBS for 1 h at 4° C.

### Click Reaction

Coverslips were incubated at room temperature for 30 min in a click reaction mixture (0.5 μM TAMRA*594, 1 mM copper(II) sulfate, 100 mM sodium ascorbate, 100 mM Tris-HCl (pH=8.0)). Cells were then washed 3 times for 15 min with 3% FBS in PBS/0.1% Triton X-100. After washes and briefly dried coverslips were mounted onto glass slides containing antifade mountant with 1 μg·mL⁻¹ DAPI.

### Example 10: Detection of SJG-136-Click in Mouse Bone Marrow by Confocal Microscopy

Mice were treated with a compound of formula (IV) according to the invention (0,3 mg/kg) during 45 minutes.

### Sample(s) Preparation and Fixation

The bone(s) (i.e. femur and tibia) were dissected from the mouse and surrounding muscle(s) were removed. Said bone(s) were immediately fixed with 4% ice-cold PFA solution for 24 hours at 4° C. in a 15 mL conical tube. The bone sample(s) were then washed three times with PBS. Each wash was carried out for 5 minutes at 4° C. with 7 mL of PBS under constant agitation.

### Decalcification and Cryoprotection

A DCAL solution, containing 0.5 M of EDTA, was prepared by dissolving 186.1 g of EDTA in 800 mL of ddH₂O with the addition of about 20 g of NaOH pellets to bring the mixture to a pH in the range of 7.4-7.6 by continuous stirring. The volume is adjusted to 1 L and the solution is filtered on a 0.5 μm filter.

A cryoprotectant (CPT) solution (1×) was also prepared by dissolving 200 g of sucrose and 20 g of polyvinylpyrrolidone (PVP) in 700 mL of PBS under constant stirring by a magnetic stirrer. The volume was then adjusted to 1 L and the solution was mixed until a homogeneous solution was obtained.

After complete removal of PBS, 10 mL of the ice-cold DCAL solution were added to the sample(s) and the mixture was left to incubate at 4° C. for 24 hours on a rotator with constant agitation. The bone(s) were washed three times with PBS. Each wash was carried out for 5 minutes at 4° C. with 7 mL of PBS under constant shaking.

After complete removal of PBS, 10 mL of the ice-cold CPT solution were added, and the bone(s) were incubated for 24 hours at 4° C. The CPT was then removed before immediately proceeding to embedding.

### Embedding and Cryosectioning

An embedding (EBM) solution was prepared, freshly before embedding, by dissolving 8 g of gelatin, 2 g of PVP and 20 g of sucrose in 80 mL of PBS at 65° C. while slowly stirring. The volume of the solution was then adjusted to 100 mL and left under stirring until it became clear. Then, stirring was stopped and the solution was left to incubate at 60° C. until all the bubbles disappeared.

5 mL of the EBM solution were added, and the mixture was incubated for 45 min at 60° C. in a water bath.

A few drops of the EBM solution were added to a tissue mold. The bone(s) sample(s) were then placed in the EBM solution and the tissue mold was filled up completely with said EBM solution. No bubbles were formed during this step.

The sample(s) were incubated at room temperature for 30 minutes to allow the EBM to solidify completely. Then, the mold was stored at −80° C. Once the tissue mold froze, it was precooled at −23° C. for 60 min in a cryostat before sectioning. The solid block of EBM was removed from the mold and glued to the holder using OCT tissue embedding medium.

Tissue sections were cut with a thickness of 10 μm at −23° C. using a microtome blade.

While sectioning, each new section was transferred to a microscope slide kept at room temperature by touching the slide to the tissue. The temperature difference between the sections and slides promoted stronger adhesion.

The tissue sections were then dried at room temperature for 30 minutes.

### Immunostaining

A permeabilization solution was prepared by adding 0.1 mL of Triton X-100 to 99.9 mL of PBS and dissolving it completely.

A blocking solution was prepared by dissolving 3 g of bovine serum albumin (BSA) in 100 mL of PBS under constant stirring by a magnetic stirrer.

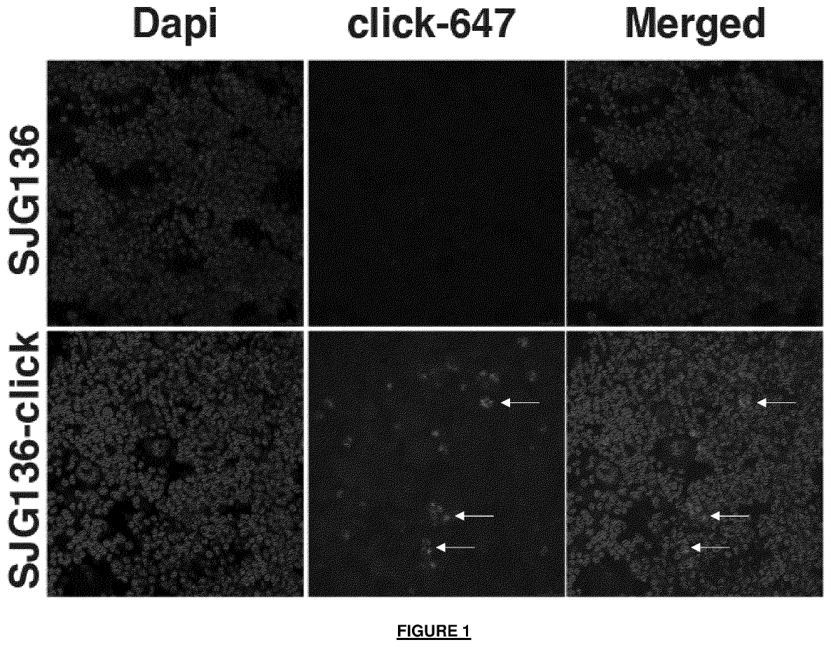

500 μL of a click-it solution were prepared 15 minutes before use by adding in 430 μL of PBS: 20 μL of CuSO₄ at 100 mM, 1.2 μL of Alexa Fluor™ 647 azide (also called herein and in FIG. 1 “click-647”), sold by the company Thermo Fisher, at 1mM and 50 μL of ascorbate acid at 1 M in this order.

At room temperature, margins were drawn around the tissue section on the same slide of the glass slide where the tissue section was attached using a PAP pen and let to dry completely. The section was rehydrated by adding 200 μL of PBS and incubated at room temperature for 5 minutes. The PBS was discarded and 200 μL of a permeabilization solution were added at room temperature for 10 minutes.

200 μL of a freshly prepared blocking solution were added and the mixture incubated for 30 minutes at room temperature. The blocking solution was then removed and 200 μL of click-it solution were immediately added. The mixture was then protected from light and incubated for 30 minutes at room temperature. After the incubation, the click-it solution was removed and the section washed with three baths of PBS 3% BSA for 5 minutes at room temperature.

### Nuclear Staining

200 μL of a freshly prepared DNA staining solution (DAPI (4′,6-diamino-2-phenylindole)) were added over the tissue section and incubated for 10 minutes at room temperature.

The staining solution was then removed and the section washed three times with PBS at room temperature.

### Mounting

The PBS was discarded from the sample(s) and a few drops of mounting medium (ProLong antifade mountant sold by the company Thermo Fisher) were immediately added. A clean coverslip was then slowly placed on top of the tissue section.

### Confocal Imaging

The prepared slide were placed on the microscope stage and the results of the confocal imaging observation are reported in FIG. 1.

### Results

Three columns are represented in FIG. 1 illustrating various comparative staining of sample(s).

(i) In the left-handed column, the staining with DAPI of the nucleus of the sample(s)'s cells are represented as positive control.

The top image of this column represents the imaging result obtained when the bone marrow samples were treated with DAPI and the compound SJG136, outside of the invention.

The bottom image of this column represents the imaging result obtained when the bone marrow samples were treated with DAPI and the compound SJG136-click (corresponding to the compound of formula (IV) of the invention).

The images obtained in these two experiments are similar and the nucleus of the bone marrow cells are clearly visible.

(ii) The column of the middle of FIG. 1 represents the imaging results obtained when the samples of this experiments were treated with click-647 and SJG136, outside of the invention (top image) or with click-647 and SJG136-click, according to the invention (bottom image), both in the absence of DAPI.

While nothing is visible in the top image of this column, i.e. when the samples were treated with click-647 and SJG136, the treatment with click-647 and SJG136-click leads to the observation, as represented in the bottom image of this column, of several light spots, clearly distinct one from the others, which represent sites of reticulation by the SJG136-click, linked to click-647. White arrows have been added to pinpoint some of these spots.

(iii) In the right-handed column of FIG. 1:
the top image is the result of merging the top images of the first and second column and is identical to the image obtained when the sample(s) are only treated with DAPI and SJG136 (left-handed-column, top image).
the bottom image is the result of merging the bottom images of the first and second column.

Accordingly, in this image, the nucleus of the cells of the sample(s) are all clearly visible as well as the light spots observed in the bottom image of the middle column. This merged image confirms that all the light spots observed are within the nucleus of cells of the treated sample(s) and accordingly are as expected sites of reticulation by the SJG136-click linked to click-647.

1. A compound of formula (I) 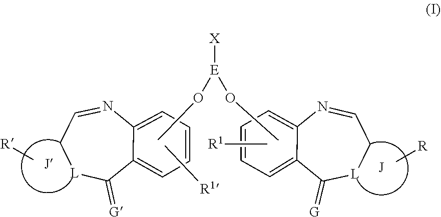
wherein:
X is a C₂-C₇ azide or alkyne group,
E is a C₃-C₁₂ alkyl group, or a group of the following formula: 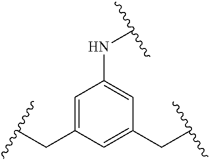 [SMILES: *Cc1cc(C*)cc(N*)c1]
wherein the label identifies the bond to the radical X;
G is an oxygen atom or a CH₂ group,
L is a nitrogen atom or a CH group,
J is a saturated or unsaturated, mono- or polycondensed C₅-C₆ heterocycloalkyl group containing nitrogen atom,
R is selected from the group consisting in hydrogen atom; halogen atom, and in particular a fluorine atom; C₂-C₃ alkynyl group, and in particular a propynyl group, substituted by a secondary amine; methylidene group; C₁-C₃ alkenyl group, optionally substituted by one or several halogen atoms, in particular by fluorine atoms, or a secondary amine; C₁-C₃ alkyl group; phenyl group and carbonyl group,
R¹ is selected from the group consisting in hydrogen atom; C₁-C₃ alkyl group; C₁-C₃ alkoxy group optionally substituted by a secondary amine or an ethynyloxy group, and
wherein G′, J′, R′ and R¹′ are independently chosen from the same groups as their corresponding counterparts G, J, R and R¹.

2. The compound according to claim 1, being of formula (II): 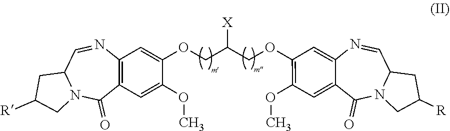
wherein:
m′ and m″ are integers independently ranging from 1 to 10 and their sum is less than 12, and
X, R and R′ are as defined in claim 1.

3. The compound according to claim 1, being of formula (III): 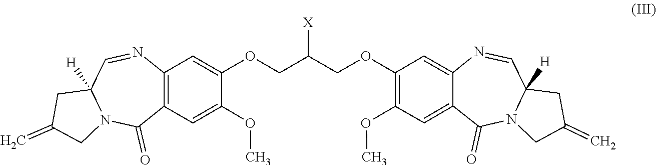
wherein X is as defined in claim 1.

4. The compound according to claim 3, wherein X is an alkyne and is in particular of formula (IV):

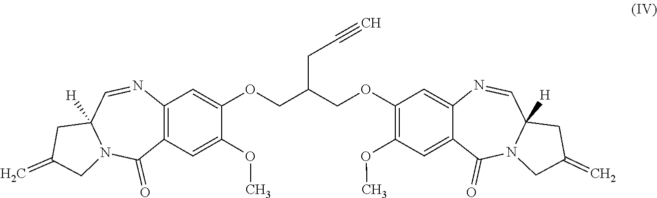

5. A kit comprising at least one compound according to claim 1, and at least one label bearing a group complementary to the X radical of said compounds for a “click-chemistry” reaction, the label being in particular a fluorescent label or a biotinylated label

6. (canceled)

7. An in-vitro or ex vivo method for visualizing DNA crosslinks in cells, the method comprising:
(a) having at least one cell,
(b) contacting said cell with at least one compound of formula (I) defined in claim 1 under conditions allowing said compound to induce DNA crosslinks in the cell,
(c) contacting the cell obtained in step (b) with at least one label bearing a group which is complementary for a click-chemistry reaction to the X radical of the said compound of formula (I), under conditions allowing the reaction of click-chemistry between the said X radical and the complementary group,
(d) detecting the label in the cell obtained at step (c).

8. An in vitro or ex vivo method for assessing the resistance or sensitivity of a tumor in a patient to a crosslinking agent, in particular to a PBD dimer, and more particularly to a compound of formula (I) as defined in claim 1, comprising at least the steps of:
(a) having at least one cell from the said tumor,
(b) contacting said cell with at least one said compound of formula (I) under conditions allowing said compound to induce DNA crosslinks in the cell,
(c) contacting the cell obtained in step (b) with at least one label bearing a group which is complementary for a click-chemistry reaction to the X radical of the compound of formula (I) under conditions allowing the reaction of click-chemistry between the said X radical and the complementary group,
(d) measuring the labeling in the cell obtained at step (c), and
(e) optionally comparing the labeling measured at step (d) to a reference level.

9. An in vitro or ex vivo method for identifying or screening a candidate molecule and/or a candidate treatment for its ability to improve the efficiency of a crosslinking agent, in particular of a PBD dimer, notably of a compound of formula (I) as defined in claim 1, comprising at least the steps of:
(a) having at least one cell, in particular a tumor cell,
(b) contacting said cell with at least one candidate molecule and/or applying a candidate treatment to said cell,
(b′) contacting said cell with at least one compound of formula (I) as defined in claim 1, under conditions allowing said compound to induce DNA crosslinks in the cell,
(c) contacting the cells obtained after the previous steps with a label bearing a group which is complementary to the X radical of the said compound of formula (I), under conditions allowing the reaction of click-chemistry between the said X radical and the complementary group,
(d) measuring the labeling in the cell obtained at step (c), and
(e) comparing the intensity of the labeling obtained at step (d) to a reference labeling intensity obtained when the method is performed in the absence of the candidate molecule and/or of the candidate treatment,
wherein said steps (b) and (b′) can occur simultaneously or sequentially, preferably in that order.

10. The method according to claim 7, further comprising at least one washing step, particularly at least one washing step after each step ranging from (a) to (d), in particular from (b) to (c).

11. The method according to claim 7, further having an intermediate step between step (b) and (c) wherein the cell is permeabilized and then fixed.

12. The method according to claim 7, wherein the label is a fluorescent label.

13. A process for preparing a compound according to claim 2, comprising at least the steps of:
reacting a pyrrolidine derivative of formula (A): 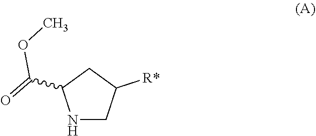
wherein R* can be R or R′ as defined in claim 1, when R* is R, the chiral carbon in (A) is in the (S) configuration and when R* is R′, said chiral carbon is in the (R) configuration,
with a pyrrolobenzodiazepine dimer core of formula (B): 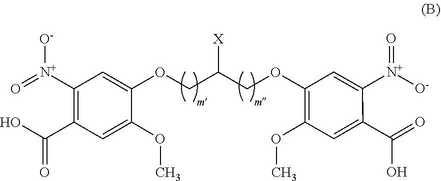
to form a bis-nitroamide of formula (C): 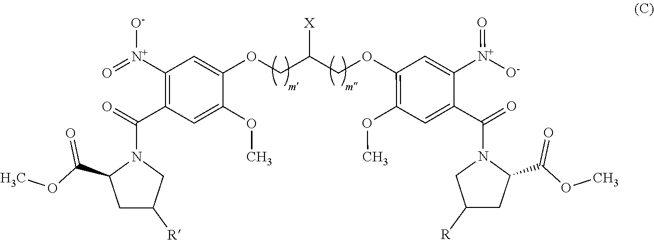
promoting the cyclisation of the so-obtained compound (C) to form a compound of formula (D): 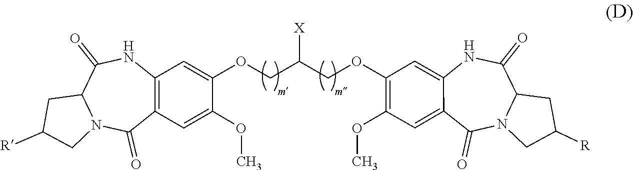
and
converting the compound (D) to form a compound according to claim 2 by protecting the amide nitrogen, reducing the carbonyl, then performing a deprotection and subsequent dehydration step, wherein R, R′, X, m′ and m″ are as defined in claim 2.

14. The process of claim 13 wherein the obtained compound is a compound of formula

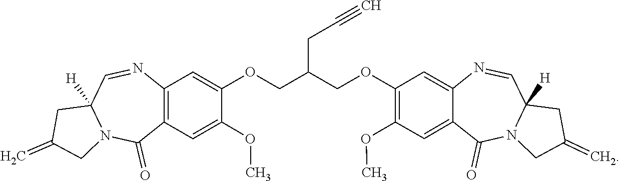 [SMILES: C#CCC(COc1cc2c(cc1OC)C(=O)N1CC(=C)C[C@H]1C=N2)COc1cc2c(cc1OC)C(=O)N1CC(=C)C[C@H]1C=N2]

15. A compound having a formula selected from the group consisting of: 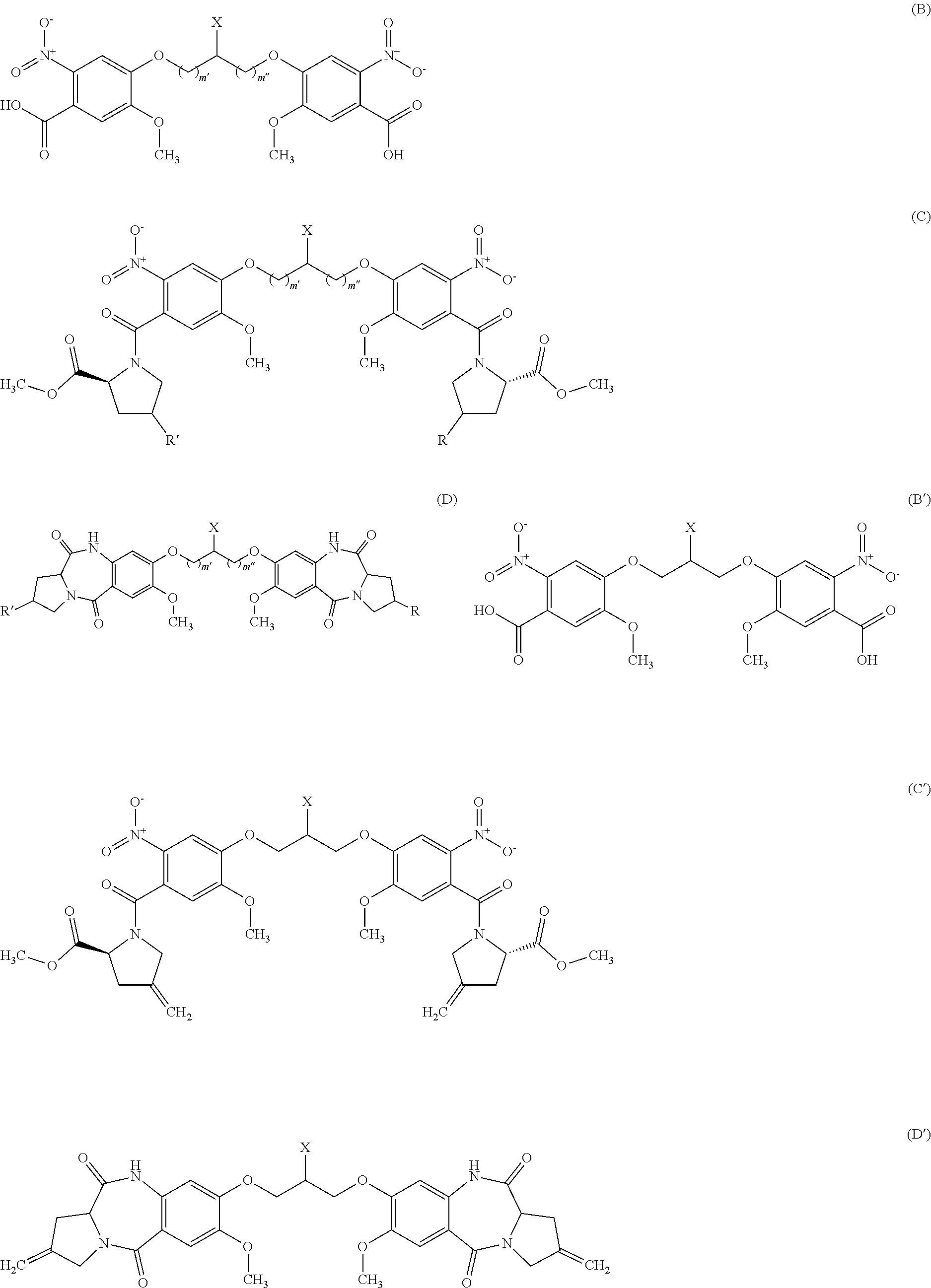
wherein:
m′ and m″ are integers independently ranging from 1 to 10 and their sum is less than 12, and
X, R and R′ are as defined in claim 1.

16. The method according to claim 8, further comprising at least one washing step, particularly at least one washing step after each step ranging from (a) to (d), in particular from (b) to (c).

17. The method according to claim 8, further having an intermediate step between step (b) and (c) wherein the cell is permeabilized and then fixed.

18. The method according to claim 8, wherein the label is a fluorescent label.

19. The method according to claim 9, further comprising at least one washing step, particularly at least one washing step after each step ranging from (a) to (d), in particular from (b) to (c).

20. The method according to claim 9, further having an intermediate step between step (b) and (c) wherein the cell is permeabilized and then fixed.

21. The method according to claim 9, wherein the label is a fluorescent label.
